(19) United States
(12) Patent Application Publication
(10) Pub. No.: US 2007/0110728 A1
(43) Pub. Date: May 17, 2007

(54) Method for preventing and treating diabetes using dg119

(75) Inventors: Matthias Austen, Goettingen (DE); Daria Onichtchouk, Gottingen (DE); Thomas Siegmund, Landolfshausen (DE); Kristin Aduroja, Dusseldorf (DE); Bettina Rudolph, Hannover (DE); Friedrich Harder, Gottingen (DE)

Correspondence Address: ROTHWELL, FIGG, ERNST & MANBECK, P.C., 1425 K STREET, N.W., SUITE 800, WASHINGTON, DC, 20005, US

(73) Assignee: DEVELOGEN AKTIENGESELLSCHAFT, Goettingen (DE)

(21) Appl. No.: 10/580,564

(22) PCT Filed: Nov. 29, 2004
(86) PCT No.: PCT/EP04/13535

§ 371 Date: May 26, 2006

(30) Foreign Application Priority Data
Nov. 28, 2003 (EP) 03027514.3

(51) Int. Cl. C12N 5/08; A61K 38/17; A61K 35/39

(52) U.S. Cl. 424/93.7; 435/366; 514/12

(57) ABSTRACT

The present invention relates generally to methods for preventing and/or treating pancreatic disorders, particularly those related to diabetes, by administering a DG119-1 product or a DG119-1 agonist and/or an antagonist To a DG 119-2 product.

The present invention relates generally to methods for preventing and/or treating pancreatic disorders, particularly those related to diabetes, by administering a DG119product and/or an agonist or an antagonist thereof.

### BACKGROUND OF THE INVENTION

The pancreas is an exocrine gland that secretes digestive enzymes directly into the digestive tract as well as an endocrine gland that secretes hormones into the blood stream. The exocrine function is assured by acinar and centroacinar cells that secrete various digestive enzymes via intercalated ducts into the duodenum. The functional unit of the endocrine pancreas is the islet of Langerhans. Islets are scattered throughout the exocrine portion of the pancreas and are composed of four main cell types: alpha-, beta-, delta- and PP-cells (reviewed for example in Kim & Hebrok, 2001, Genes Dev. 15: 111-127). Beta-cells produce insulin, represent the majority of the endocrine cells and form the core of the islets, while alpha-cells secrete glucagon and are located in the periphery. Delta-cells and PP-cells are less numerous and secrete somatostatin and pancreatic polypeptide, respectively. Recently, cells producing the neuropeptide Ghrelin have been found in pancreatic islets (Wierup et al., 2002, Regul Pept. 107:63-9.).

Early pancreatic development has been well studied in different species, including chicken, zebrafish, and mice (for a detailed review, see Kim & Hebrok, 2001, supra). The pancreas develops from distinct dorsal and ventral anlagen. Pancreas development requires specification of the pancreas anlage along both anterior-posterior and dorsal-ventral axes. A number of transcription factors, that are critical for proper pancreatic development have been identified (see Kim & Hebrok, 2001, supra; Wilson et al., 2003, Mech Dev. 120: 65-80).

In humans, the acinar and ductal cells retain a significant proliferative capacity that can ensure cell renewal and growth, whereas the islet cells become mostly mitotically inactive. This is in contrast to rodents where beta-cell replication is an important mechanism in the generation of new beta cells. It has been suggested, that during embryonic development, pancreatic islets of Langerhans originate from differentiating duct cells or other cells with epithelial morphology (Bonner-Weir & Sharma, 2002, J Pathol. 197: 519-526; Gu et al., 2003, Mech Dev. 120: 35-43). In adult humans, new beta-cells arise in the vicinity of ducts (Butler et al., 2003, Diabetes 52: 102-110; Bouwens & Pipeleers 1998, Diabetologia 41: 629-633). However, also an intra-islet location or an origin in the bone marrow has been suggested for precursor cells of adult beta-cells (Zulewski et al., 2001, Diabetes 50: 521-533; Ianus et al., 2003, J Clin Invest. 111: 843-850). Pancreatic islet growth is dynamic and responds to changes in insulin demand, for example during pregnancy or due to changing body weight during childhood. In adults, there is a good correlation between body mass and islet mass (Yoon et al., 2003, J Clin Endocrinol Metab. 88: 2300-2308).

Blood vessels play an important role in organ development (Matsumoto et al., 2001, Science 294: 559-563; Lammert et al., 2003, Mech Dev. 120(1): 59-64). For the formation of pancreatic buds from primitive endoderm, the presence of adjacent blood vessels is necessary (Lammert et al., 2001, Science 294: 564-567). The authors also show that blood vessels can induce the expression of insulin in primitive endoderm in tissue conjugation experiments. Furthermore, mice genetically engineered to develop additional blood vessels in their pancreas show a greatly increased islet number. There is a close association between duct-like structures and blood vessels in fetal mouse pancreas, suggesting that blood vessels may play an important role in beta cell neogenesis from ducts (reviewed in Cleaver & Melton, 2003, Nat Med. 9(6):661-668). Thus, it is of great importance to identify signals produced by endothelial cells which can regulate or induce the generation of functional cell types during embryogenesis and/or adult regeneration. Of particular interest are signals which control the formation of new pancreatic beta cells due to their potential relevance for diabetes therapies.

Pancreatic beta-cells secrete insulin in response to rising glucose levels and other secretagogues such as arginine. Insulin amongst other hormones plays a key role in the regulation of the fuel metabolism. Insulin leads to the storage of glycogen and triglycerides and to the synthesis of proteins. The entry of glucose into muscles and adipose cells is stimulated by insulin. In patients who suffer from diabetes mellitus type I or LADA (latent autoimmue diabetes in adults (Pozzilli & Di Mario, 2001, Diabetes Care. 8:1460-67) beta-cells are destroyed due to autoimmune attack. The amount of insulin produced by the remaining pancreatic islet cells is too low, resulting in elevated blood glucose levels (hyperglycemia). In diabetes type II, liver and muscle cells loose their ability to respond to normal blood insulin levels (insulin resistance). High blood glucose levels (and also high blood lipid levels) in turn contribute to an impairment of beta-cell function and to an increase in beta-cell apoptosis. It is interesting to note that the rate of beta-cell neogenesis does not appear to change significantly in type II diabetics (Butler et al., 2003 supra), thus causing a reduction in total beta-cell mass over time. Eventually the application of exogenous insulin becomes necessary in type II diabetics.

Improving metabolic parameters such as blood sugar and blood lipid levels (e.g. through dietary changes, exercise, medication or combinations thereof) before beta-cell mass has fallen below a critical threshold leads to a relatively rapid restoration of beta-cell function. However, even after such a treatment the pancreatic endocrine function would remain impaired due to the only slightly increased regeneration rate. Treatments which increase the rate of neogenesis will have a beneficial effect due to enhanced insulin secretory capacity.

In type I diabetics, where beta-cells are being destroyed by autoimmune attack, treatments have been devised which modulate the immune system and may be able to stop or strongly reduce islet destruction (Raz et al., 2001, Lancet 358: 1749-1753; Chatenoud et al., 2003, Nat Rev Immunol. 3: 123-132; Homann et al., Immunity. 2002, 3:403-15). However, due to the relatively slow regeneration of human beta-cells such treatments can only be successful if they are combined with agents that can stimulate beta-cell regeneration.

A variety of model organisms has been used to study the formation of beta cells and to analyze the effect of treatments aimed at the improvement of diabetic conditions. Zebrafish has become a popular model vertebrate for the study of developmental processes as well as for pharmacological and toxicological studies over the last decade (Rubinstein, 2003, Curr Opin Drug Discov Devel. 6(2):218-23; Grunwald & Eisen, 2002, Nat Rev Genet. 3(9): 717-24). In this organism, large numbers of transparent embryos which rapidly develop outside of their mother are readily available. Transgenic lines expressing fluorescent proteins under the control of tissue-specific promoters allow to rapidly assess the effects of pharmacological treatments or gene loss- and gain-of-function treatments. Zebrafish islets contain the same cell-types in a similar spatial organization as mammalian islets. A large number of genes which control pancreatic development in mammals also control pancreatic development in zebrafish (Biemar et al., 2001, Dev Biol. 230(2): 189-203; Ober et al., 2003, Mech Dev. 120(1): 5-18). Suppressing gene function in zebrafish embryos using antisense oligonucleotides, modified Peptide Nucleic Acids (mPNAs) or other antisense compounds with good efficiency and specificity yields phenotypes which are usually indistinguishable from genetic mutants in the same gene (Nasevicius et al., Nat Genet. 2000 26(2):216-20; Effimov et al., NAR 26; 566-575; Urtishak et al., 5th international conference on zebrafish development and genetics, Madison/Wisc. 2002, abstr. #17). Thus, zebrafish embryos represent a relevant model to identify genes or compounds which control beta cell formation in humans.

Diabetes is a very disabling disease, because today's common anti-diabetic drugs do not control blood sugar levels well enough to completely prevent the occurrence of high and low blood sugar levels. Chronically elevated blood sugar levels are toxic and cause long-term complications such as renopathy, retinopathy, neuropathy, and peripheral vascular disease. There is also a host of related conditions, such as obesity, hypertension, heart disease, and hyperlipidemia, for which persons with diabetes are substantially at risk.

Apart from the impaired quality of life for the patients, the treatment of diabetes and its long term complications presents an enormous financial burden to our healthcare systems with rising tendency. Thus, for the prevention or treatment of diabetes mellitus type I, LADA, and diabetes mellitus type II there is a strong need in the art to identify factors that induce regeneration of pancreatic insulin producing beta-cells.

### SUMMARY OF THE INVENTION

In this invention, we disclose a novel and so far unknown use for DG119 proteins as factors secreted from blood vessels within the pancreas to regulate endocrine development. The DG119 proteins regulate or induce the generation of functional cell types during embryogenesis and/or adult regeneration. DG119 proteins control the formation of new pancreatic beta cells.

In particular, DG119-1 is disclosed as positive regulator of insulin-producing cells. Thus, DG119-1 stimulates the formation or regeneration of insulin producing cells, particularly beta-cells. Thus, DG 119-1 and certain modulators (e.g. agonists, stimulators) can be used in the treatment and/or prevention of diseases caused by, accompanied by and/or associated with dysfunctions of pancreatic cells, particularly pancreatic beta-cells, such as diabetes mellitus type I, LADA, and diabetes mellitus type II.

In another embodiment of this invention, DG119-2 is disclosed herein as negative regulator of insulin-producing cells. Thus, certain modulators (e.g., antagonists, Inhibitors) of DG119-2 stimulate the formation or regeneration of insulin producing beta-cells and can be used in the treatment and/or prevention of diseases caused by, accompanied by and/or associated with dysfunctions of pancreatic cells, particularly pancreatic beta-cells, such as diabetes mellitus type I, LADA, and diabetes mellitus type II.

More particularly, the present invention provides methods for treating patients suffering from a disease linked to functionally impaired and/or reduced numbers of pancreatic islet cells, particularly insulin producing beta-cells, by administering a therapeutically effective amount of a DG119-1 product or an agonist/stimulator thereof and/or an antagonist/inhibitor of a DG119-2 product. Functional impairment or loss of pancreatic islet cells may be due to autoimmune attack in diabetes type I or LADA, or due to cell degeneration in progressed diabetes type II. The methods of the present invention may also be used to treat patients at risk to develop degeneration of insulin producing beta-cells to prevent the start or progress of such a process.

Numerous additional aspects and advantages of the invention will become apparent to those skilled in the art upon consideration of the following description of the Figures and detailed description of the invention which describes presently preferred embodiments thereof.

### BRIEF DESCRIPTION OF THE FIGURES

FIG. 1 shows internal organs of 17 day old zebrafish embryos. The fish embryos carry a reporter construct consisting of a fluorescent protein cDNA functionally linked to insulin promoter sequences. Bright green fluorescence in these fish marks regions of endogenous zebrafish insulin expression. At 17 days of development, a zebrafish pancreas includes one bigger islet and several smaller islets (see arrowheads). Also shown are the gall bladder and the gut of the 17 day old fish embryos.

FIG. 2 shows that DG119-1 increases the main islet (Brockmann body) size. Main islets of five 17 day old fish embryos injected either with mRNA of mouse DG119-1 (200 μg/μl) or with control mRNA at the same concentration are shown. In DG119-1 injected embryos, the mean size of the pancreatic islets (Brockmann bodies) is significantly increased (see right panel of the figure), if compared to the size of the islets in fish where control mRNA was injected (see left panel of FIG. 2)).

FIG. 3 shows that DG119-1 increases the size of small islets. Isolated gastrointestinal tracts of five 17-day-old fish embryos injected at one-cell-stage either with mRNA of mouse DG119-1 or with control mRNA at the same concentration (200 ηg/μl) are shown. Brockmann bodies and smaller islets appear in bright green (fluorescence). On the left picture, DG119-1 injected embryos are shown, on the right picture, embryos injected with control mRNA are shown. In DG119-1 injected embryos, the size of the small pancreatic islets is significantly increased, meaning, that DG119-1 stimulates the formation/development of pancreatic islets.

FIG. 4 shows that both DG119-1 and DG119-2 are expressed in pancreatic blood vessels in mammals.

FIG. 4A shows a cryosection of a pancreas from a 17,5-day-embryonic mouse that was analysed by in situ hybridization with antisense mouse DG119-1 probe (dark blue) and stained with anti-insulin antibodies (red) and nuclear dye DAPI (light blue). DG119-1 is mainly located to pancreatic blood vessels (note anuclear erythrocytes within the vessel).

FIG. 4B shows a cryosection of a pancreas from 17,5-day embryonic mouse which was analysed by in hybridization with antisense mouse DG119-2 probe (dark). DG119-2 is located to pancreatic blood vessels, similar to DG119-1.

FIG. 5 shows that loss of DG119-2 function increases islet size and insulin expression in zebrafish.

FIGS. 5A and 5B shows zebrafish injected with DG119A. One cell stage zebrafish carrying the transgene with insulin regulatory sequences linked to a fluorescent protein cDNA were either injected with antisense oligonucleotides to DG119-2A or with control oligonucleotides. Note the increase of islet size in DG119-2A fish where the function of DG119-2A was inhibited (FIG. 5B) compared to control fish with active DG119-2A (FIG. 5A). DG119-2A is therefore inhibiting the development of pancreatic islets.

FIG. 5C shows the relative expression level of Insulin, Pdx1 and Pax4 RNA in 48 hours post fertilization (Hpf). DG119-2A (light grey columns) or control (black and white columns) antisense-injected embryos measured using quantitative RT-PCR procedure. Note that the insulin RNA content is 3 fold increased in DG119-2A antisense injected fish. FIG. 5C shows that the enlarged pancreatic islets generate significantly higher insulin levels in fish where the function of DG119-2A is inhibited completely.

FIG. 6 shows the sequences of zebrafish DG119 proteins.

FIG. 6A shows the sequence of zebrafish DG119-1A1A protein (719 amino acids, shown in the one-letter-code) (SEQ ID NO:1).

FIG. 6B. shows the sequence of zebrafish DG119-1B1B protein (594 amino acids, shown in the one-letter-code) (SEQ ID NO:2).

FIG. 6C shows the sequence of zebrafish DG119-2A2A protein (146 amino acids, shown in the one-letter-code; C-terminus incomplete) (SEQ ID NO:3).

FIG. 6D shows the sequence of zebrafish DG119-2B2B protein (287 amino acids, shown in the one-letter-code; C-terminus incomplete) (SEQ ID NO:4).

FIG. 7 shows a sequence alignment of DG119 proteins from human, mouse, and zebrafish (Mm, mouse; Hs, human; Dr; zebrafish). Please see the Examples section for more detail.

FIG. 8 shows the structure of the transgenic constructs. Shown is the shematic representation of the mouse mDG119-1 transgenic constructs.

FIG. 8A shows the rIP promoter (0.8 kb rat insulin II promoter) as a thin line, the mouse DG119-1 cDNA (mDG119-1) as white box, the hybrid-intron structure (hybrid-intron) as grey box and the polyadenylation signal (bgh-polyA) as black box.

FIG. 8B shows the Pdx1 promoter (4.3 kb mouse Pdx1-promoter) as a thin line, the mouse DG119-1 cDNA (mDG119-1) as white box, the hybrid-intron structure (hybrid-intron) as grey box and the polyadenylation signal (bgh-polyA) as black box.

FIG. 9 shows pancreatic islets of mDG119-1 transgenic mice with ectopic mDG119-1 expression. Taqman expression analysis on islet cDNA isolated from two wild type and two transgenic littermates using a mDG119-1 specific primer/probe pair. The data are presented as fold mDG119-1 induction relative to wild type mDG119-1 expression in islets.

FIG. 9A shows that pancreatic islets of transgenic mice with ectopic mDG119-1 expression (rIP-mDG119-1 islets) have a significantly higher expression of DG119-1.

FIG. 9B shows pancreatic islets of Pdx1-mDG119-1 transgenic mice with ectopic mDG119-1 expression.

FIG. 10A shows the growth curves of DG119-1 transgenic mice (rIP-mDG119-1) compared to wild type mice (wt) on HF diet. Data are presented as mean bodyweight in gram/over time ± standard deviation.

FIG. 10B shows the lean and fat body mass in rIP-mDG119-1 transgenic mice compared to wild type mice (wt) on HF diet. After 8 weeks on HF diet lean and fat body mass of individual male mDG119-1 transgenic mice (dark grey bars, N=12) and male littermate controls (light grey bars, N=14) was measured using NMR analysis. The data are expressed as mean organ weight as % of bodyweight ± standard deviation.

FIG. 11 shows body length of rIP-mDG119-1 transgenic mice compared to wild type mice (wt) on HF diet. Body length of 10 weeks old male wild type mice (light grey bar, N=12) and rIP-mDG119-1 transgenic mice (dark grey bar, N=14). The data are expressed as mean body length in cm ± standard deviation.

FIG. 12A shows random fed blood glucose levels of DG119-1 transgenic mice (rIP-mDG119-1) and wild type mice (referred to as wt). Shown are blood glucose levels from random fed male wild type mice (♦, N=15) and rIP-mDG119-1 transgenic mice (▪, N=18). The data are expressed as mean blood glucose ± standard deviation. The blood glucose levels is significantly lower in DG119-1 transgenic mice; meaning that higher expression of DG119-1 in pancreatic islets of mammals will lower the blood glucose level.

FIG. 12B shows blood glucose levels of transgenic mice (rIP-mDG119-1) that were on a starvation diet compared to wild type mice (wt) on the same diet. Shown are blood glucose levels from overnight (16h) fasted male wild type mice (♦, N=12) and rIP-mDG119-1 transgenic mice (▪, N=14). The data are expressed as mean blood glucose ± standard deviation. The blood glucose levels is significantly lower in DG119-1 transgenic mice; meaning that higher expression of DG119-1 in pancreatic islets of mammals will lower the blood glucose level, even if those mammals are on a starvation diet.

FIG. 13A shows steady state plasma insulin levels in transgenic mice (rIP-mDG119-1) compared to wild type mice (wt). Shown are plasma insulin levels from random fed male wild type mice (light grey bar, N=16) and rIP-mDG119-1 transgenic mice (dark grey bar, N=18). The data are expressed as mean plasma glucagon levels ± standard deviation. The data show that DG119-1 if expressed at higher levels in pancreatic islets of mammals lower the plasma insulin level.

FIG. 13B shows steady state plasma glucagon levels in rIP-mDG119-1 transgenic mice. Shown are plasma glucagon levels from random fed male wild type mice (light grey bar, N=13) and rIP-mDG119-1 transgenic mice (dark grey bar, N=14). The data are expressed as mean plasma glucagon levels ± standard deviation. The data show that DG119-1 if expressed at higher levels in pancreatic islets of mammals lower the plasma glucagons level.

FIG. 14A shows intraperitoneal glucose tolerance of rIP-mDG119-1 transgenic mice. Glucose was injected intraperitoneally in 5 weeks old male wild type mice (♦, N=14) and 5 weesk old male rIP-mDG119-1 transgenic mice (▪, N=17). Blood samples were collected from the tail vein and blood glucose was measured at the indicated time intervals. The data are expressed as mean blood glucose ± standard deviation. The blood glucose levels are significantly lower in mammals with higher DG119-1 expression in pancreatic islets.

FIG. 14B shows intraperitoneal insulin tolerance of rIP-mDG119-1 transgenic mice. Recombinant human insulin was injected intraperitoneally in 6 weeks old male wild type mice (♦, N=15) and 6week old male rIP-mDG119-1 transgenic mice (▪, N=16). Blood samples were collected from tail vein and blood glucose was measured at the indicated time intervals. The data are expressed as mean blood glucose as % of basal blood glucose ± standard deviation.

FIG. 15A shows pancreatic insulin content of rIP-mDG119-1 transgenic mice compared to wild type mice.

FIG. 15B shows the beta cell area of Pdx1-mDG119-1 transgenic mice compared to wild type mice (wt). Shown is the relative beta cell area expressed as ratio of total insulin area to total amylase area. The area of exocrine tissue (amylase) and beta cell area (insulin) was quantified in 96 images randomly taken from 12 paraffin sections of each wild type (wt) and Pdx1-mDG119-1 transgenic (tg) pancreata. Each image was blindly (without knowledge of the genotype) quantified twice by the same person, error bar shows standard deviation of 2 measurements. The Figure shows a high insulin content in beta cells of mammals islets in transgenic DG119-1 animals.

FIG. 15C shows the islet size of Pdx1-mDG119-1 transgenic mice compared to wild type mice (wt). Shown is that the islet size which is expressed as a ratio of islet area/islet number is five-fold larger in transgenic DG119-1 mice. Beta cell area (insulin) and the number of islets was quantified in 96 images randomly taken from 12 paraffin sections of each wild type (wt) and Pdx1-mDG119-1 transgenic (tg) pancreata. Each image was blindly (without knowledge of the genotype) quantified twice by the same person, error bar shows standard deviation of 2 measurements.

FIG. 16 shows in the left panels paraffin sections of wild type (referred to as wt in the upper left panel) or Pdx1-mDG119-1 transgenic mice (lower left panel). The sections were stained for amylase (exocrine pancreas, green) and insulin (endocrine beta cells, red) by indirect immunofluorescence using standard methods. Sections were photographed at low magnification with a Leica FLIII fluorescence stereomicroscope. Typical fields are shown. In sections of transgenic mice, notably more fields contain larger and more numberous islets. In the right panels islets were isolated from three wild type mice (referred to as wt in the upper right panel) and three transgenic rIP-mDG119-1 mice and pooled. 160 islets were removed from each pancreas for further analysis. The remaining islets were photographed and counted. Islets from transgenic mice appeared larger and more numerous (about 20% more total islets in transgenics).

stain is shown in stain is shown in of this Fig. In shows that the (is higher control (wt, wildtype)

FIG. 17 shows insulin secretion of mDG119-1 transgenic islets compared to wild type (referred to as wt) mouse islets in vitro. The two columns on the left show the insulin secretion of wt mouse islets, the two columns on the right show the insulin secretion of mDG119-1 transgenic islets. White columns show the basal insulin secretion and black columns show the glucose-stimulated insulin secretion. The figure shows that mDG119-1 transgenic islets secrete approximately twice as much insulin as wt islets and show similar inducibility.

### DESCRIPTION OF THE INVENTION

Before the present invention is described, it is understood that all technical and scientific terms used herein have the same meanings as commonly understood by one of ordinary skill in the art to which this invention belongs.

In the present invention the term “beta-cell regeneration” refers to the restoration of normal beta-cell function by increasing the number of functional insulin secreting beta-cells and/or by restoring normal function in functionally impaired beta-cells.

As used herein, the term “DG119 product” includes proteins such as purified natural, synthetic, or recombinant DG119-1 or DG119-2 and variants thereof. Variants include insertion, substitution and deletion variants and chemically modified derivatives. Variants also include recombinant proteins, for example but not limited to hybrids or fusions of DG119-1 or DG119-2 and other proteins. Also included are proteins or peptides substantially homologous to the human DG119-1 or DG119-2 protein having the amino acid sequence published as GenBank Accession Number XP_{—}034000 (DG119-1) or GenBank Accession Number NP_{—}872293 (DG119-2). The term “DG119 product” also includes nucleic acids, e.g. RNA or DNA coding for the above described DG119-1 or DG119-2 protein product. The term “DG119 product” also includes DG119-1 or DG119-2 homodimers or heterodimers of a DG119-1 or DG119-2 protein product and another protein.

The term “substantially homologous” as used herein means having a degree of homology to the biologically active DG119 protein product having the amino acid sequence published as GenBank Accession Number XP_{—}034000 (DG119-1) or GenBank Accession Number NP_{—}872293 (DG119-2), that is preferably in excess of 70%, most preferably in excess of 80%, and even more preferably in excess of 90% or 95%. The degree of homology between the mouse and the human protein is about 91%, and it is contemplated that preferred mammalian DG119 proteins will have a similarly high degree of homology. Also included are proteins which are hybrids between DG119-1 and another protein which retain the stimulatory effect on islet cell formation found in DG119-1. The percentage of homology or percent identity between a DG119 product and a human DG119 protein or nucleic acid may be determined according to standard procedures, e.g. by using the BLAST algorithm. Preferably, it is calculated as the percentage of nucleotide or amino acid residues found in the smaller of the two sequences that align with identical nucleotide or amino acid residues in the sequence being compared, when four gaps in a length of 100 nucleotides or amino acids may be introduced to assist in that alignment. Also included as substantially homologous is any DG119 protein product which may be isolated by virtue of cross-reactivity with antibodies to the DG119 protein product or whose genes may be isolated through hybridization with the gene or with segments of the gene encoding the DG119 protein product.

As used herein, the term “agonist/stimulator” of a DG119-1 product refers to any substance that is inducing or stimulating the expression and/or function of DG119-1.

As used herein, the term “antagonists to DG119-2” refers to any substance that is interfering with the expression and/or function of DG119-2. It includes any effectors or modulators of DG119-2, e.g. antagonists or inhibitors. In particular, the “antagonists to DG119-2” can be fragments or otherwise modified parts of DG119-2. Fragments for example could one of two modules of the protein; e.g. the cystein-rich domain (amino acids 30-118 in human DG119-2) or the C-terminal olfactomedin domain (amino acids 447-704 in human DG119-2) or peptides derived therefrom could be used as antagonist to DG119-2. In addition to fragmentation, antagonists to DG119-2 can also be obtained by changing single amino acids or by chemical modifications of the protein. The “antagonists to DG119-2” can include any effectors, e.g. receptors, enzymes, proteins, ligands, agents, or substrates that either directly or indirectly modulate (inhibit) the action of DG119-2 protein products. The “antagonists to DG119-2” can include effectors/modulators of DG119-2 polynucleotides and/or polypeptides, antibodies, biologically active nucleic acids, such as antisense molecules, RNAi molecules or ribozymes, aptamers, peptides or low-molecular weight organic compounds recognizing said DG119-2 polynucleotides or polypeptides.

The term “biologically active” as used herein means that the DG119-1 protein product or an agonist thereof stimulates and/or induces the differentiation of insulin producing cells and/or promotes the protection, survival, or regeneration of islet cells.

In connection with the present invention, the term, progenitor cells' relate to undifferentiated cells capable of being differentiated into insulin producing cells. The term particularly includes stern cells, i.e. undifferentiated or immature embryonic, adult, or somatic cells that can give rise to various specialized cell types. The term “stem cells” can include embryonic stem cells (ES) and primordial germ cells (EG) cells of human or animal origin. Isolation and culture of such cells is well known to those skilled in the art (see, for example, Thomson et al., 1998, Science 282:1145-1147; Shamblott et al., 1998, Proc. Natl. Acad. Sci. USA 95:13726-13731; U.S. Pat. No. 6,090,622; U.S. Pat. No. 5,914,268; WO 0027995; Notarianni et al., 1990, J. Reprod. Fert. 41:51-56; Vassilieva et al., 2000, Exp. Cell. Res. 258:361-373). Adult or somatic stem cells have been identified in numerous different tissues such as intestine, muscle, bone marrow, liver, and brain. WO 03/023018 describes a novel method for isolating, culturing, and differentiating intestinal stem cells for therapeutic use. In the pancreas, several indications suggest that stem cells are also present within the adult tissue (Gu & Sarvetnick, 1993, Development 118:3346; Bouwens, 1998, Microsc Res Tech 43:332-336; Bonner-Weir, 2000, J. Mol. Endocr. 24:297-302).

Embryonic stem cells can be isolated from the inner cell mass of pre-implantation embryos (ES cells) or from the primordial germ cells found in the genital ridges of post-implanted embryos (EG cells). When grown in special culture conditions such as spinner culture or hanging drops, both ES and EG cells aggregate to form embryoid bodies (EB). EBs are composed of various cell types similar to those present during embryogenesis. When cultured in appropriate media, EB can be used to generate in vitro differentiated phenotypes, such as extraembryonic endoderm, hematopoietic cells, neurons, cardiomyocytes, skeletal muscle cells, and vascular cells. We have previously described a method that allows EB to efficiently differentiate into insulin-producing cells (as described in patent application PCT/EP02/04362, published as WO 02/086107 and by Blyszczuk et al., 2003, Proc Natl Acad Sci USA. 100(3):998-1003, which are incorporated herein by reference).

In this invention, we disclose a novel and so far unknown use for DG119-1 to stimulate and/or induce the formation or regeneration of insulin producing beta-cells and thus, a use in the treatment and prevention of diseases going along with impaired beta-cell function, for example but not limited to diabetes mellitus.

The present invention is based on the finding that DG119-1 stimulates the formation of insulin producing islets. Furthermore, this invention discloses that antagonists to DG119-2 stimulate the formation of islets that are insulin producing. Thus, a therapeutically effective amount of DG119-1 product or an agonist thereof or an antagonist to a DG119-2 product may be administered to promote the regeneration of pancreatic beta-cells or to promote the differentiation or formation of insulin-producing cells from progenitor cells in vitro or in vivo. The present invention further relates to applications in the medical field that directly arise from the method of the invention. Additionally, the present invention relates to applications for the identification and characterization of compounds with therapeutic medical effects or toxicological effects that directly arise from the method of the invention.

According to this invention the DG119-1 product or agonist or the antagonist to a DG119-2 product may be administered
i) as a pharmaceutical composition e.g. enterally, parenterally or topically, preferably directly to the pancreas,
ii) via implantation of DG119-1 protein product or antagonist to DG119-2 product expressing cells, and/or
iii) via gene therapy
   as described in more detail below.

Further, the DG119-1 or DG119-2 expression level or function in a patent might be influenced by a pharmaceutically active substance administered
i) as a pharmaceutical composition e.g. enterally, parenterally or topically, preferably directly to the pancreas,
ii) via cell based therapy and/or
iii) via gene therapy
   as described in more detail below.

The DG119-1 product or the pharmaceutically active substance influencing the DG119-1 or DG119-2 expression level or function may be administered in the above described manner alone or in combination with another pharmaceutical composition useful to treat beta-cell degeneration, for example hormones, growth factors or immune modulating agents.

A DG119-1 product or an agonist thereof or an antagonist of a DG119-2 product may be administered in patients suffering from a disease going along with impaired beta-cell function, for example but not limited to diabetes type I, LADA, or progressed diabetes type II. It is further contemplated that the above compounds may be administered preventively to patients at risk to develop beta-cell degeneration, like for example but not limited to patents suffering from diabetes type II or LADA in early stages. A variety of pharmaceutical formulations and different delivery techniques are described in further detail below.

The invention further comprises the use of cells with activated pancreatic genes, e.g. as described in WO 03/023018, which is herein incorporated by reference. Examples of preferred pancreatic genes are Pdx1, Pax4, Pax6, neurogenin3 (ngn3), Nkx6.1, Nkx6.2, Nkx2.2, HB9, Beta2/NeuroD, Isl1, HNF1-alpha, HNF-1 beta and HNF3 of human or animal origin. The pancreatic genes may be introduced into the cells by transfection or transduction, e.g. transfection of progenitor or stem cells or transduction of pancreatic duct and islet cells (Noguchi H., et al., 2003, Diabetes 52: 1732-1737).

DG119 products, e.g. DG119 protein products, are preferably produced via recombinant techniques because such methods are capable of achieving high amounts of protein at a great purity, but are not limited to protein products expressed in bacterial, plant, mammalian, or insect cell systems.

DG119 Protein Product

Recombinant DG119-1 or DG119-2 protein product forms include glycosylated and non-glycosylated forms of the protein. In general, recombinant techniques involve isolating the genes encoding for DG119-1 or DG119-2 protein product, cloning the gene in suitable vectors and/or cell types, modifying the gene if necessary to encode a desired variant, and expressing the gene in order to produce the DG119-1 or DG119-2 protein product.

Alternatively, a nucleotide sequence encoding the desired DG119-1 or DG119-2 protein product may be chemically synthesized. It is contemplated that a DG119-1 or DG119-2 protein product may be expressed using nucleotide sequences that vary in codon usage due to the degeneration of the genetic code or allelic variations or alterations made to facilitate production of the protein product by the selected cell.

The DG119-1 or DG119-2 protein products according to this invention may be isolated or generated by a variety of means. Exemplary methods for producing DG119 protein products, vectors, host cells, and culture growth conditions for the expression of DG119-1 or DG119-2 protein, as well as methods to synthesize variants of DG119-1 or DG119-2 protein product are known to those skilled in the art.

DG119-1 or DG119-2 protein product variants are prepared by introducing appropriate nucleotide changes into the DNA encoding the polypeptide or by in vitro chemical synthesis of the desired polypeptide. It will be appreciated by those skilled in the art that many combinations of deletions, insertions, and substitutions can be made resulting in a protein product variant presenting DG119-1 or DG119-2 biological activity. Mutagenesis techniques for the replacement, insertion or deletion of one or more selected amino acid residues are well known to one skilled in the art DG119-1 or DG119-2 substitution variants have at least one amino acid residue of the human or mouse DG119-1 or DG119-2 amino acid sequence removed and a different residue inserted in its place. Such substitution variants include allelic variants, which are characterized by naturally occurring nucleotide sequence changes in the species population that may or may not result in an amino acid change.

Chemically modified derivatives of DG119-1 or DG119-2 protein products also may be prepared by one of skill in the art given the disclosures herein. The chemical moieties most suitable for derivatization include water soluble polymers. A water soluble polymer is desirable because the protein to which it is attached does not precipitate in an aqueous environment, such as a physiological environment. Preferably, the polymer will be pharmaceutically acceptable for the preparation of a therapeutic product or composition. One skilled in the art will be able to select the desired polymer based on such considerations as whether the polymer/protein conjugate will be used therapeutically, and if so, the desired dosage, circulation time, resistance to proteolysis, and other considerations. A particularly preferred water-soluble polymer for use herein is polyethylene glycol. Attachment at residues important for receptor binding should be avoided if receptor binding is desired. One may specifically desire an N-terminal chemically modified protein.

The present invention contemplates use of derivatives which are prokaryote-expressed DG119-1 or DG119-2, or variants thereof, linked to at least one polyethylene glycol molecule, as well as use of DG119-1 or DG119-2, or variants thereof, attached to one or more polyethylene glycol molecules via an acyl or alkyl linkage.

The present invention also discloses use of derivatives which are prokaryote-expressed DG119-1 or DG119-2, or variants thereof, linked to at least one hydrophobic residue, for example fatty acid molecule, as well as use of DG119-1 or DG119-2 9, or variants thereof, attached to one or more hydrophobic residues. For example, patent application published as WO 03/010185, which is hereby incorporated by reference, describes a method for producing acylated polypeptides in transformed host cells by expressing a precursor molecule of the desired polypeptide which are then to be acylated in a subsequent in vitro step.

Polynucleotides Encoding DG119 Protein Product

The present invention further provides polynucleotides that encode DG119-1 or DG119-2 protein products whether recombinantly produced or naturally occurring.

A nucleic acid sequence encoding a DG119-1 or DG119-2 protein product, can readily be obtained in a variety of ways, including, without limitation, chemical synthesis, cDNA or genomic library screening, expression library screening, and/or PCR amplification of cDNA. These methods and others useful for isolating such nucleic acid sequences are set forth, for example, by Sambrook et al. (Molecular Cloning: A Laboratory Manual, Cold Spring Harbor Laboratory Press, Cold Spring Harbor, N.Y., 1989), by Ausubel et al., eds (Current Protocols in Molecular Biology, Current Protocols Press, 1994), and by Berger and 2 5 Kimmel (Methods in Enzymology: Guide to Molecular Cloning Techniques, vol. 152, Academic Press, Inc., San Diego, Calif., 1987). Chemical synthesis of a nucleic acid sequence which encodes a DG119 protein product can also be accomplished using methods well known in the art, such as those set forth by Engels et al. (Angew. Chem. Intl. Ed., 28:716-734, 3 0 1989).

Included within the scope of this invention are DG119-1 or DG119-2 product polynucleotides with the native signal sequence and other pre-pro sequences as well as polynucleotides wherein the native signal sequence is deleted and replaced with a heterologous signal sequence. The heterologous signal sequence selected should be one that is recognized and processed, i.e., cleaved by a signal peptidase, by the host cell. For prokaryotic host cells that do not recognize and process the native DG119-1 or DG119-2 signal sequence, the signal sequence may be substituted by a prokaryotic signal sequence selected, for example, from the group of the alkaline phosphatase, penicillinase, or heat-stable enterotoxin 11 leaders. For yeast secretion, the native DG119-1 or DG119-2 signal sequence may be substituted by the yeast invertase, alpha factor, or acid phosphatase leaders. In mammalian cell expression the native signal sequence is satisfactory, although other mammalian signal sequences may be suitable. Expression and cloning vectors generally include a nucleic acid sequence that enables the vector to replicate in one or more selected host cells.

DG119 Pharmaceutical Compositions

Pharmaceutical compositions comprising a DG119-1 product or an agonist thereof or an antagonist to a DG119-2 product typically include a therapeutically effective amount of the active ingredient in admixture with one or more pharmaceutically and physiologically acceptable formulation. In addition to the active ingredients, pharmaceutical compositions may contain suitable pharmaceutically acceptable carriers comprising excipients and auxiliaries, which facilitate processing of the active compounds into preparations, which can be used pharmaceutically. Further details on techniques for formulation and administration may be found in the latest edition of Remington's Pharmaceutical Sciences (Maack Publishing Co., Easton, Pa.), the disclosure of which is hereby incorporated by reference.

Once the therapeutic composition has been formulated, it may be stored in sterile vials as a solution, suspension, gel, emulsion, solid, or dehydrated or lyophilized powder. Such formulations may be stored either in a ready to use form or in a form, e.g., lyophilized, requiring reconstitution prior to administration. The optimal pharmaceutical formulations will be determined by one skilled in the art depending upon considerations such as the route of administration and desired dosage. Such formulations may influence the physical state, stability, rate of in vivo release, and rate of in vivo clearance of the present DG119-1 or DG119-2 proteins, variants and derivatives. Other effective administration forms, such as slow-release formulations, inhalant mists, or orally active formulations are also envisioned. For example, in a sustained release formulation, the active ingredient may be bound to or incorporated into particulate preparations of polymeric compounds (such as polylactic acid, polyglycolic acid, etc.) or liposomes.

Administration/Delivery

The active ingredient may be administered by any suitable means, e.g. enterally or parenterally or topically directly to the pancreas, as known to those skilled in the art. The specific dose may be calculated according to considerations of body weight, body surface area or organ size. Further refinement of the calculations necessary to determine the appropriate dosage for treatment involving each of the above mentioned formulations is routinely made by those of ordinary skill in the art and is within the ambit of tasks routinely performed. Appropriate dosages may be ascertained through use of the established assays for determining dosages utilized in conjunction with appropriate dose-response data. The final dosage regimen involved in a method for treating the above described conditions will be determined by the attending physician, considering various factors which modify the action of drugs, e.g., the age, condition, body weight, sex and diet of the patient, the severity of any infection, time of administration and other clinical factors. As studies are conducted, further information will emerge regarding the appropriate dosage levels for the treatment of various diseases and conditions.

It is envisioned that the continuous administration or sustained delivery of the active ingredient may be advantageous for a given treatment. While continuous administration may be accomplished via a mechanical means, such as with an infusion pump, it is contemplated that other modes of continuous or near continuous administration may be practiced. For example, chemical derivatization or encapsulation may result in sustained release forms of the protein having the effect of continuous presence, in predictable amounts, based on a determined dosage regimen. Thus, the active ingredients include proteins derivatized or otherwise formulated to effectuate such continuous administration.

Cell therapy, e.g. pancreatic implantation of cells producing DG119-1 protein product or antagonists to DG119-2 protein product, is also contemplated. This embodiment would involve implanting cells capable of synthesizing and secreting a biologically active form of DG119-1 protein product or antagonists to DG119-2 protein product into patients. Such DG119-1 protein product or antagonists to DG119-2 protein product-producing cells may be cells that are natural producers of DG119-1 protein product or antagonists to DG119-2 protein product or may be cells that are modified to express such proteins. Modified cells include recombinant cells whose ability to produce a DG119-1 protein product or an antagonist to DG119-2 protein product has been augmented by transformation with a gene encoding the desired DG119-1 protein product or an antagonist to a DG119-2 product in a vector suitable for promoting its expression and secretion. In order to minimize a potential immunological reaction in patients being administered DG119-1 product or an antagonist to a DG119-2 product of a foreign species, it is preferred that the cells be of human origin and produce a human DG119-1 protein product or human antagonist to a DG119-2 protein product. Likewise, it is preferred that recombinant cells are transformed with an expression vector containing a gene encoding a human DG119-1 protein product or with an inhibitory nucleic acid for the DG119-2 product, e.g. an antisense molecule, a ribozyme or an RNAi molecule or a DNA molecule coding therefor. Implanted cells may be encapsulated to avoid infiltration of surrounding tissue. Human or nonhuman animal cells may be implanted in patients in biocompatible, semipermeable polymeric enclosures or membranes that allow release of the active ingredient but that prevent destruction of the cells by the patient's immune system or by other detrimental factors from the surrounding tissue.

Alternatively, cells may be introduced into a patient in need intraportally via a percutaneous transhepatic approach using local anaesthesia. Such surgical techniques are well known in the art and can be applied without any undue experimentation, see Pyzdrowski et al, 1992, New England J. Medicine 327:220-226; Hering et al., Transplantation Proc. 26:570-571, 1993; Shapiro et al., New England J. Medicine 343:230-238, 2000.

Further, the invention relates to a cell preparation comprising differentiated stem cells exhibiting insulin production, e.g. an insulin-producing cell line obtainable by the method described above. The insulin-producing cells may exhibit a stable or a transient expression of at least one gene involved in beta-cell differentiation. The cells are preferably human cells that are derived from human stem cells. For therapeutic applications the generation of autologous human cells from adult stem cells of a patient is especially preferred. However, the insulin producing cells may also be derived from nonautologous cells. If necessary, immune rejection may be avoided by encapsulation, immunosuppression and/or modulation or due to non-immunogenic properties of the cells. The insulin producing cells preferably exhibit characteristics that closely resemble naturally occurring beta-cells, e.g. an enhanced insulin production by a factor of at least 2, preferably at least 3 after addition of 27.7 mM glucose.

The cell preparation of the invention is preferably a pharmaceutical composition comprising the cells together with pharmacologically acceptable carriers, diluents and/or adjuvants. The pharmaceutical composition is preferably used for the prevention or treatment of pancreatic diseases, e.g. diabetes.

According to the present invention, the functional cells treated with a DG119-1 product or an agonist thereof or an antagonist to a DG119-2 product are transplanted preferably intrahepatic, directly into the pancreas of an individual in need, or by other methods. Alternatively, such cells are enclosed into implantable capsules that can be introduced into the body of an individual, at any location, more preferably in the vicinity of the pancreas, or the bladder, or the liver, or under the skin. Methods of introducing cells into individuals are well known to those of skill in the art and include, but are not limited to, injection, intravenous or parenteral administration. Single, multiple, continuous or intermittent administration can be effected. The cells can be introduced into any of several different sites, including but not limited to the pancreas, the abdominal cavity, the kidney, the liver, the celiac artery, the portal vein or the spleen. The cells may also be deposited in the pancreas of the individual.

The methodology for the membrane encapsulation of living cells is familiar to those of ordinary skill in the art, and the preparation of the encapsulated cells and their implantation in patients may be accomplished without undue experimentation. See, e.g. U.S. Pat. Nos. 4,892,538, 5,011,472, and 5,106.627, each of which is specifically incorporated herein by reference. A system for encapsulating living cells is described in PCT Application WO 91/10425 of Aebischer et al., specifically incorporated herein by reference. See also, PCT Application WO 91/10470 of Aebischer et al., Winn et al., Exper. Neurol., 1 13:322-3)29, 1991, Aebischer et al., Exper. Neurol., 11 1:269-275, 1991; Tresco et al., ASAIO, 38:17-23, 1992, each of which is specifically incorporated herein by reference. Techniques for formulating a variety of other sustained- or controlled-delivery means, such as liposome carriers, bio-erodible particles or beads and depot injections, are also known to those skilled in the art.

In another embodiment gene therapy ex vivo is envisioned, i.e. the patient's own cells may be transformed ex vivo to produce DG119-1 protein product or a protein stimulating DG119-1 expression or a protein or a substance, e.g. an inhibitory nucleic acid, inhibiting DG119-2 expression and would be directly reimplanted. For example, cells retrieved from the patient may be cultured and transformed with an appropriate vector. After an optional propagation/expansion phase, the cells can be transplanted back into the same patient's body, particularly the pancreas, where they would produce and release the desired protein product. Delivery by transfection and by liposome injections may be achieved using methods, which are well known in the art. Any of the therapeutic methods described above may be applied to any suitable subject including, for example, mammals such as dogs, cats, cows, horses, rabbits, monkeys, and most preferably, humans.

DG119-1 product gene therapy in vivo is also envisioned, by introducing the gene coding for DG119-1 protein product into targeted pancreas cells via local injection of a nucleic acid construct or other appropriate delivery vectors. (Hefti, J. Neurobiol., 25:1418-1435, 1994). For example, a nucleic acid sequence encoding a DG119-1 protein product may be contained in an adenoassociated virus vector or adenovirus vector for delivery to the pancreas cells. Alternative viral vectors include, but are not limited to, retrovirus, herpes simplex virus and papilloma virus vectors. Physical transfer, either in vivo or ex vivo as appropriate, may also be achieved by liposome-mediated transfer, direct injection (naked DNA), receptor-mediated transfer (ligand-DNA complex), electroporation, calcium phosphate precipitation or microparticle bombardment (gene gun).

Immunosuppressive drugs, such as cyclosporin, can also be administered to the patient in need to reduce the host reaction versus graft. Allografts using the cells obtained by the methods of the present invention are also useful because a single healthy donor could supply enough cells to regenerate at least partial pancreas function in multiple recipients.

The DG119 nucleic acids and proteins and effectors/modulators thereof may be administered either as a monotherapy or as a combination therapy with other pharmaceutical agents. For example, they may be administered together with at least one other pharmaceutical agent suitable for the treatment or prevention of pancreatic diseases and/or obesity and/or metabolic syndrome, particularly with at least one pharmaceutical agent suitable for stimulating and/or inducing the differentiation or development of insulin producing cells from progenitor cells. Further, they may be administered together with at least one pharmaceutical agent which has an immunosuppressive activity, e.g. antibodies, polypeptides and/or peptidic or non-peptidic low molecular weight substances. Preferred examples of immunosuppressive agents are listed in the following Table 1.

TABLE 1
Exemplary agents for immune suppression

| Names | Mechanism |
|---|---|
| 2-amino-1,3-propanediol | Used for preventing or treating chronic rejection in a patient |
| derivatives | receiving an organ or tissue allo- or xenotransplant |
| 2-amino-2[2-(4-octylphenyl)ethyl] | Immunosuppression, from accelerated lymphocyte homing |
| propane-1,3-diol hydrochloride | |
| 40-O-(2-hydroxyethyl)-rapamycin, | Sirolimus (rapamycin) derivative, used for acute kidney |
| SDZ-RAD, Everolimus | rejection; reduces rejection and graft vasculopathy following |
| | heart transplantation by inhibiting cell proliferation |
| 6-(3-dimethyl-aminopropionyl) | Immunosuppressing action useful also for treating autoimmune |
| forskolin | disease |
| 6-mercaptopurine (6-MP) | Used to treat Crohn's disease, inflammatory bowel disease and |
| | for organ transplant therapy |
| A-420983 | Lck-inhibitor |
| ABX-CBL (CBL-1) | Mouse monoclonal AB targeted against human T-cell, B cells, |
| | NK cells and monocytes, for treatment of steroid-resistant graft |
| | vs host diseases, potential use in treatment of inflammatory |
| | and autoimmune disorders |
| Alefacept (human LFA-3 IgG1 | Knocks out causative memory T-lymphocytes; Used to treat |
| fusion protein) | psoriasis, a T-cell mediated inflammatory disorder |
| Antisense ICAM-1 inhibitor (ISIS | Mouse monoclonal AB blocks white blood cell adhesion to T- |
| 2302), Enlimomab, BIRR1, | cell surface molecule (ICAM-1r); treatment of kidney transplant |
| Alicaforsen | rejection |
| Antithymocyte immunoglobulin | Anti-human thymocyte, immunoglobulin; used in reversal of |
| (ATGAM) | acute kidney transplant rejection and will likely be used off- |
| | label for transplant induction therapy |
| Azathioprine | Treatment of rheumatoid arthritis and prevention of kidney |
| | transplant rejection, and other autoimmune or inflammatory |
| | disorders such as inflammatory bowel disease |
| Baohuoside-1 | Flavonoid; inhibits lymphocyte activation; Ma et al., |
| | Transplantation 78: 831-838, (2004) |
| basiliximab | Monoclonal AB that binds to receptor sites on T-cells, |
| | preventing activation by transplanted tissue (renal transplant) |
| BMS-279700 | Lck-inhibitor |
| BTI-322 | Mouse derived monoclonal AB targeted to CD2 receptor; used |
| | for prevention of first-time kidney rejection, and treatment of |
| | resistant rejection |
| Cladribine | Antimetabolite and immunosuppressive agent that is relatively |
| | selective for lymphocytes; used to treat lymphoid malignancies, |
| | e.g., hairycell leukemia |
| CP-690550 | JAK-3 inhibitor |
| Cyclophosphamide (CTX) | Immunosuppressant for treatment of arthritis and other auto- |
| | immune disorders and cancers |
| Cyclosporine (cyclosporin A, | 11 amino acid cyclic peptide; blocks helper T-cell, |
| cyclosporin) | immunosuppressant used in organ transplant therapy and |
| | other immune diseases |
| Daclizumab, HAT (Humanized Anti- | Monoclonal AB inhibits binding of IL-2 to IL-2 receptor by |
| Tac), SMART anti-Tac, anti-CD25, | binding to IL-2 receptor; suppresses T-cell activity against |
| and humanized anti-IL2-receptor | allografts (renal transplant) |
| Dexamethasone (Decadron, | An adrenocorticoid, effective immunosuppressant in various |
| Dexone, Dexasone) | disorders |
| DIAPEP-277 | Immunomodulatory properties |
| Dipeptide Boronic Acid (DPBA) | Proteasome inhibitor; Wu et al., Transplantation 78: 360-366, |
| | (2004) |
| Docosahexaenoic acid (DHA) | Immunosuppressant that lowers the proportion of T cells |
| | expressing CD4 or CD8, blocks antigen recognition process; |
| | Taku et al., Journal of Agricultural and Food Chemistry 48: |
| | 1047, 2000 |
| efalizumab | T-cell modulator that target T-cells through interactions with |
| | adhesion molecules on endothelial cell surface, target |
| | migration of T-cells into the skin and target activation of T-cells; |
| | Used to treat Psoriasis |
| Efomycine M | Leukocyte adhesion inhibitor, |
| | Anti-Inflammatory |
| FTY720 (oral myriocin derivative) | Alters lymphocyte infiltration into grafted tissues; used for |
| | prevention of organ rejection in kidney transplants |
| Glatiramer acetate (co-polymer-1) | Synthetic peptide copolymer; decoy that mimics structure of |
| | myelin so immune cells bind Copaxone instead of myelin; for |
| | multiple sclerosis |
| Glial fibrillary acidic protein (GFAP) | Possesses immunosuppressive activities in diabetic animal |
| | models; Winer et al., Nature Medicine 9: 198, (2003) |
| Gusperimus (15-deoxyspergualin) | Intravenous immunosuppressant; suppresses production of |
| | cytotoxic T-cells, neutrophils and macrophages |
| HLA-B2702 peptide | Human peptide, blocks action of NK cells and T-cell mediated |
| | toxicities, used for prevention of first kidney allograft rejection |
| hu1124 (anti-CD11a) | Humanized monoclonal antibody, targets CD11a receptor on |
| | surface of T cells to selectively inhibit immune system rejection |
| | of transplanted organs |
| hOKT31γ(Ala-Ala) | non Fc-binding humanized anti CD3 antibody |
| Infliximab | Monoclonal AB, binds and inactivates human TNFalpha; used |
| | to treat Crohn's disease and rheumatoid arthritis |
| Interferon | Immunomodulatory properties |
| ISAtx247 | Used to treat autoimmune diseases such as rheumatoid |
| | arthritis and psoriasis |
| Isotretinoin | Immunosuppressant, reduces ability of T cells to proliferate in |
| | response to immune challenge. Vergelli et al., |
| | Immunopharmacology, 31: 191, (1997) |
| L-683, 742: also described as 31- | Treatment of autoimmune diseases, infectious diseases and/or |
| desmethoxy-31-hydroxy-L-683,590 | prevention of organ transplant rejections |
| Leflunomide (ARAVA) | Antiinflammatory agent |
| Medi-500 (Ti10B9) | Intravenous monoclonal AB that targets human T-cells; treats |
| | acute kidney rejection and graft-vs-host disease |
| Medi-507 | Intravenous humanized AB directed against CD2 T-cell; used |
| | to treat corticosteroidresistant graft vs host disease and |
| | prevention of kidney rejection |
| Methotrexate | Antimetabolite used to treat Crohn's disease, severe psoriasis, |
| | and adult rheumatoid arthritis (and as an anti-cancer drug) |
| Mitoxantrone | Antiproliferative effect on cellular immune system including T- |
| | cells, B-cells and macrophages; used to treat hormone- |
| | refractory prostate cancer, acute myelogenous leukemia and |
| | multiple sclerosis |
| mycophenolate mofetil | Proliferation of T and B lymphocytes by blocking the synthesis |
| | of purine nucleotides; used in organ transplant therapy and |
| | inflammatory bowel disease |
| OKT4A | Mouse monoclonal AB targeted against human CD4 T cell; |
| | used for prevention of kidney transplant rejection when used in |
| | combination with other immunosuppressant drugs |
| Muromonab-CD3 | Monoclonal AB that binds to receptor sites on T-cells, |
| | preventing activation by transplanted tissue |
| Prednisolone | Corticosteroid, suppresses inflammation associated with |
| | transplant rejection |
| Psora-4 | Kv1.3-blocker |
| Rifampicin | Antibiotic; has immunomodulatory properties |
| Rituximab | CD20 antibody |
| S100β | possesses immunosuppressive activities in diabetic animal |
| | models |
| Sirolimus, Rapamycin | Immunosuppressant and potent inhibitor of cytokine (e.g. IL-2)- |
| | dependent T-cell proliferation (kidney transplant) |
| Tacrolimus (Prograf; FK-506) | Interferes with IL-2 TCR communication |
| Triptolide | Small molecule; Inhibits T-cell activation |

The combination therapy may comprise coadministration of the medicaments during the treatment period and/or separate administration of single medicaments during different time intervals in the treatment period.

In one embodiment of the invention, administration of a DG119-1 product and/or an agonist thereof or an antagonist of a DG119-2 product in a pharmaceutical composition to a subject in need thereof, particularly a human patent, leads to an at least partial regeneration of pancreatic cells. Preferably, these cells are insulin producing beta-cells that will contribute to the improvement of a diabetic state. With the administration of this composition e.g. on a short term or regular basis, an increase in beta-cell mass can be achieved. This effect upon the body reverses the condition of diabetes partially or completely. As the subject's blood glucose homeostasis improves, the dosage administered may be reduced in strength. In at least some cases further administration can be discontinued entirely and the subject continues to produce a normal amount of insulin without further treatment. The subject is thereby not only treated but could be cured entirely of a diabetic condition. However, even moderate improvements in beta-cell mass can lead to a reduced requirement for exogenous insulin, improved glycemic control and a subsequent reduction in diabetic complications. In another example, the compositions of the present invention will also have efficacy for treatment of patients with other pancreatic diseases such as pancreatic cancer, dysplasia, or pancreatitis, if beta-cells are to be regenerated.

In a further embodiment, the present invention allows the production of cells for the identification and/or characterisation of compounds which stimulate beta-cell differentiation, insulin secretion and/or glucose response, more particularly of compounds which increase the DG119-1 and/or decrease the DG119-2 expression level or function. This method is particularly suitable for in vivo testing for diagnostic applications and drug development or screening. The compound of interest is added to suitable cells and DG119-1 and/or DG119-2 expression or function is determined. Alternatively, a compound of interest is added to a DG119-1, DG119-1 agonist or DG119-2 antagonist treated cell and the effect on cell differentiation and/or insulin production is determined. Preferably, differentiated insulin-producing cells are used. Insulin levels in treated cells can be determined, e.g. quantified by Enzyme Linked Immunoabsorbent Assay (ELISA) or Radio Immuno Assay (RIA). Using this method, a large number of compounds can be screened and compounds that increase DG119-1 expression or support the activity of DG119-1 or compounds that the decrease DG119-2 expression or inhibit the activity of DG119-2 leading to beta-cell differentiation and/or an increase insulin secretion can be identified readily.

In a high-throughput screening method, the cells are transfected with a DNA construct, e.g. a viral or non-viral vector containing a reporter gene, e.g. the lacZ gene or the GFP gene, under regulatory control of a promoter of a gene involved in beta-cell differentiation, e.g. preferably a Pax4 promoter. The transfected cells are divided into aliquots and each aliquot is contacted with a test substance, e.g. candidate 1, candidate 2, and candidate 3. The activity of the reporter gene corresponds to the capability of the test compound to induce beta-cell differentiation.

In a further embodiment (which may be combined with the high-throughput screening as described above) a medium throughput validation is carried out. Therein, the test compound is added to stem cells being cultivated and the DG119-1 and/or DG119-2 expression and/or the insulin production is determined. Following an initial high throughput assay, such as the cell based assay outlined above where e.g. a Pax4 promoter is used as marker for beta-cell regeneration, the activity of candidate molecules to induce beta-cell differentiation is tested in a validation assay comprising adding said compounds to the culture media of the embryoid bodies. Differentiation into insulin-producing cells is then evaluated, e.g. by comparison to wild type and/or Pax4 expressing cells to assess the effectiveness of a compound.

### EXAMPLES

A better understanding of the present invention and of its many advantages will be had from the following examples, given by way of illustration.

### Example 1

### Identification of DG119 Homologues

DG119 homologous proteins and nucleic acid molecules coding therefore are obtainable from vertebrate species, e.g. mammals or fish. Particularly preferred are nucleic acid molecules and proteins encoded thereby comprising human DG119-1 and DG119-2. Human DG119-1 protein was published with GenBank Accession number XP_{—}034000; the corresponding nucleic acid was published with GenBank Accession number XM_{—}034000. Human DG119-2 protein was published with GenBank Accession number NP_{—}872293; the corresponding nucleic acid was published with GenBank Accession number NM_{—}182487. Mouse DG119-1 protein was published with GenBank Accession number NP_{—}796042; the corresponding nucleic acid was published with GenBank Accession number NM_{—}177068. Mouse DG119-2 protein was published with GenBank Accession number NP_{—}766442; the corresponding nucleic acid was published with GenBank Accession number NM_{—}172854.

To identify possible zebrafish ortologues of human and mouse DG119-1 and DG119-2, sequence databases (NCBI non redundant protein database [ftp://ftp.ncbi.nih.gov/blast/db], EST section of NCBI genbank (see Boguski et al., 1993, Nat Genet. 4(4):332-3). dbEST-database for “expressed sequence tags”], and zebrafish genome draft assembly 2 [http://www.ensembl.org/Danio rerio/]) were searched using the blastall programm (version 2.2.6, Altschul et al. 1997, Nucleic Acids Res. 25:3389-3402). Starting from the blast hits candidate genes were assembled and translated as necessary using the programms genewise (version 2.2.0, see http://www.ebi.ac.uk/Wise2/), getorf, est2genome and showseq (from the EMBOSS package version 2.7.1, see http://www.hgmp.mrc.ac.uk/Software/EMBOSS/). The resulting candidate protein sequences were compared to similar mouse and human proteins in multiple alignments made with the clustalw programm (version 1.83, see Thompson et al., 1994, Nucleic Acids Research, 22:4673-4680) to verify the homology to mouse DG119. If available from the assembly data, translation start sites were selected for antisense oligonucleotides targeting. Otherwise splice donor sites identified by alignment of zebrafish EST data or mouse protein data to zebrafish genomic sequence were used for antisense oligonucleotides targeting.

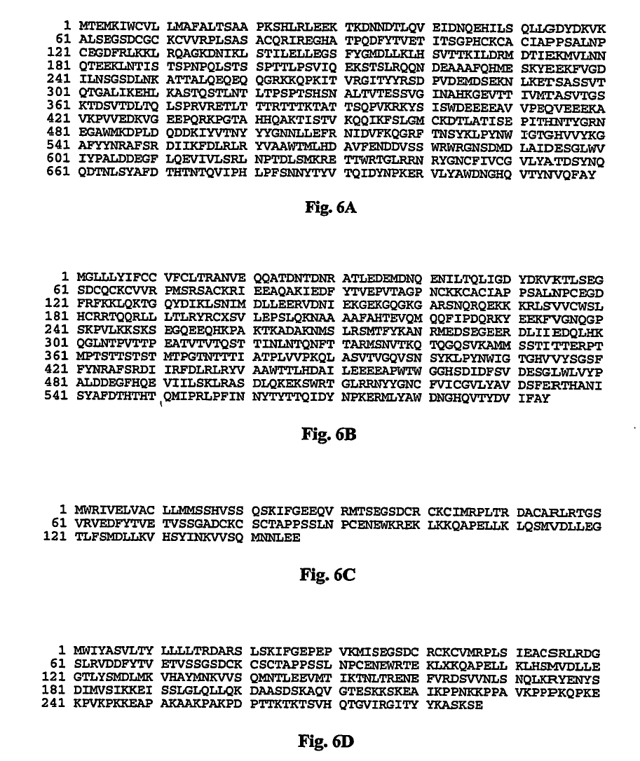

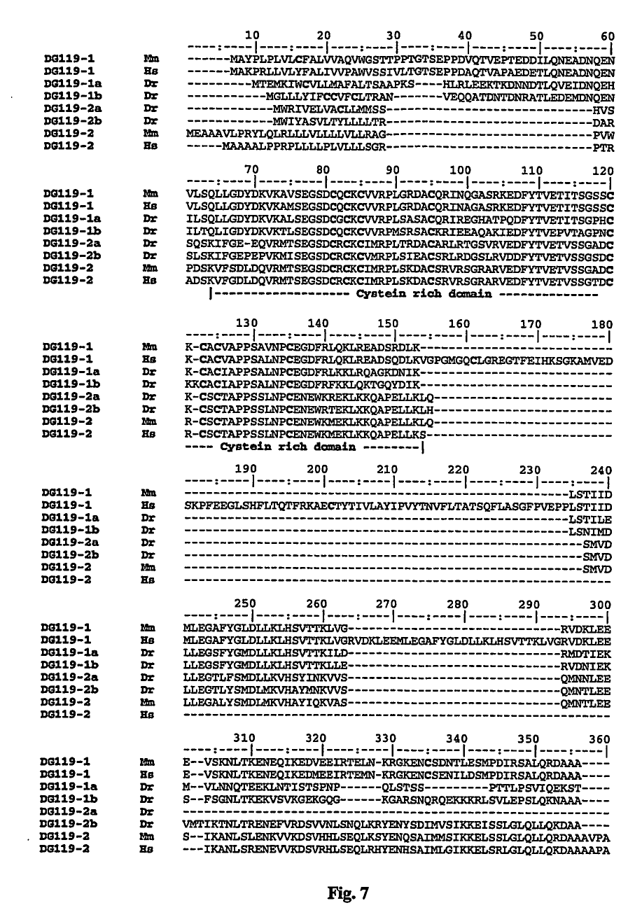

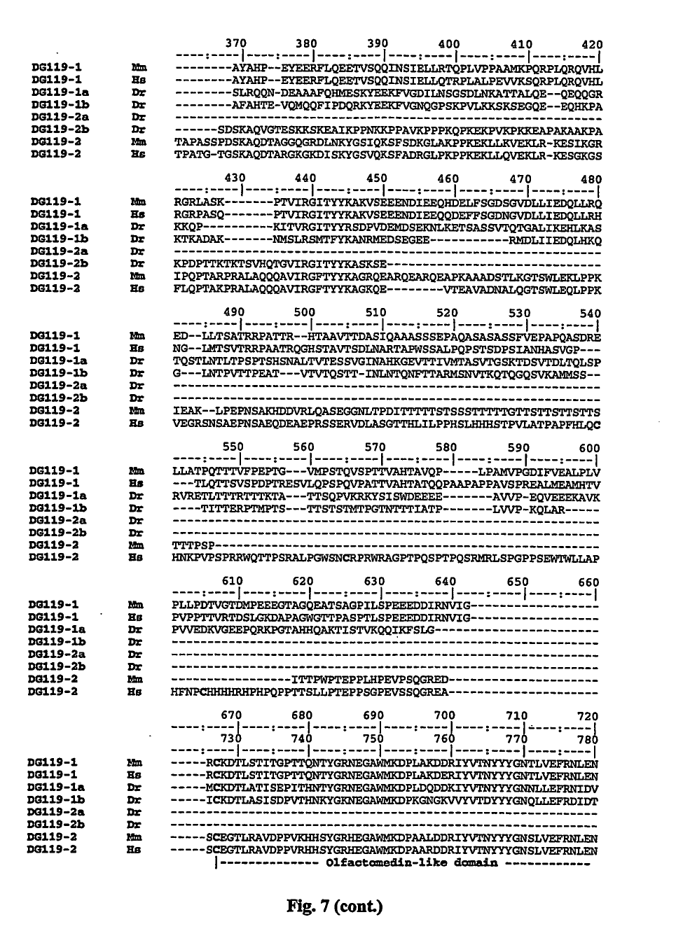

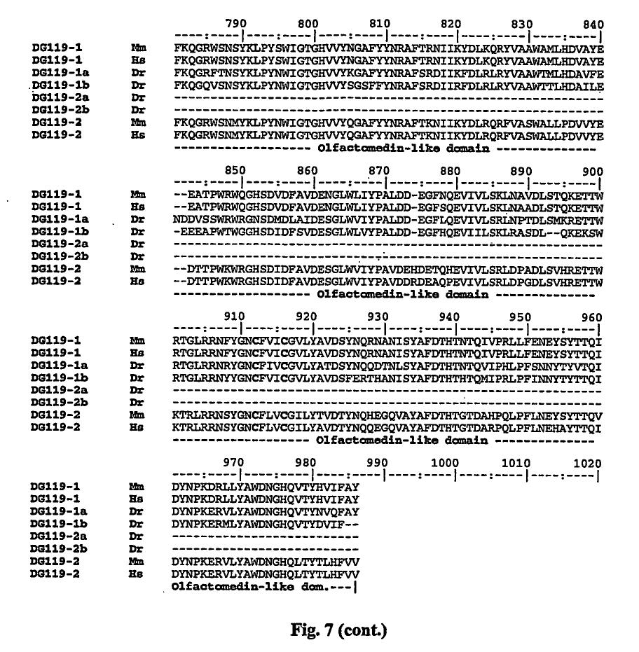

The zebrafish DG119-1A, DG19-1B, DG119-2A, and DG119-2B sequences are shown in FIG. 6A-D, respectively. An alignment of DG119 from fish, mouse, and human is shown in FIG. 7. DG119-1 Mm refers to mouse DG119-1 protein (GenBank Acc. No. NP_{—}796042), DG119-1 Hs refers to human DG119-1 protein (GenBank Acc. No. XP_{—}034000) DG119-1a Dr refers to zebrafish DG119-1a protein (SEQ ID NO:1, see FIG. 6A), DG119-1b Dr refers to zebrafish DG119-1b protein (SEQ ID NO:2, see FIG. 6B), DG119-2a Dr refers to zebrafish DG119-2a protein (SEQ ID NO:3, see FIG. 6C), DG119-2b Dr refers to zebrafish DG119-2b protein (SEQ ID NO:4, see FIG. 6D), DG119-2 Mm refers to mouse DG119-2 protein (GenBank Acc. No. NP_{—}766442), and DG119-2 Hs refers to the human DG119-2 protein (GenBank Acc. No. NP_{—}872293).

### Example 2

### Loss-of-Function Experiments in Zebrafish

To study to effect of DG119 on pancreatic function, several experiments were performed in zebrafish. Zebrafish were raised, maintained, and crossed as described (see, Westerfield, 1995, The Zebrafish Book. Eugene, Oreg.: Univ.of Oregon Press). Staging was performed according to Kimmel et al., 1995, Dev Dyn 203:253-310. Development of zebrafish embryos was carried out at 28 degrees Celsius. The age of embryos is indicated as hours post fertilization (hpf), the age of larvae as days post fertilization (dpf). Zebrafish carrying the transgene with insulin regulatory sequences linked to a fluroscent protein cDNA were used for the experiments. As control, progeny of crosses between AB and TL strain fish (Westerfield et al., supra; http://www.zfin.org) were used for injections.

DG119-1 or DG119-2A or control antisense oligonucleotide were injected into fertilized one-cell stage embryos as described (see, for example, Nasevicius & Ekker, 2000, Nat Genet 26:216-220; Urtishak et al., 2003, Dev Dyn. 228(3):405-413). Injected embryos were analysed at different stages of development or processed for quantitative RT-PCR at 48 hours post fertilization (hpf).

Images of zebrafish embryos were taken using a MZFLIII stereomicroscope (Leica) equipped with epifluorescence. In some cases, two images were taken at the same focal plane in transmitted light and using an EGFP filter, and then superimposed and processed using the Adobe Photoshop program.

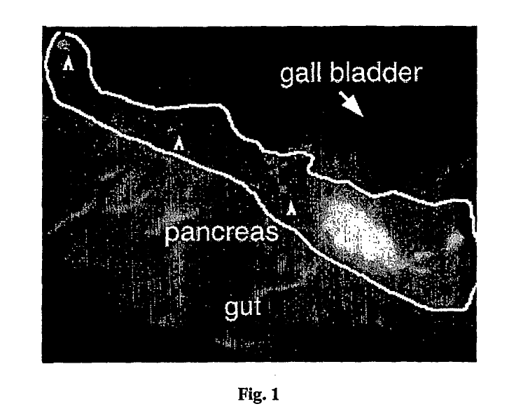

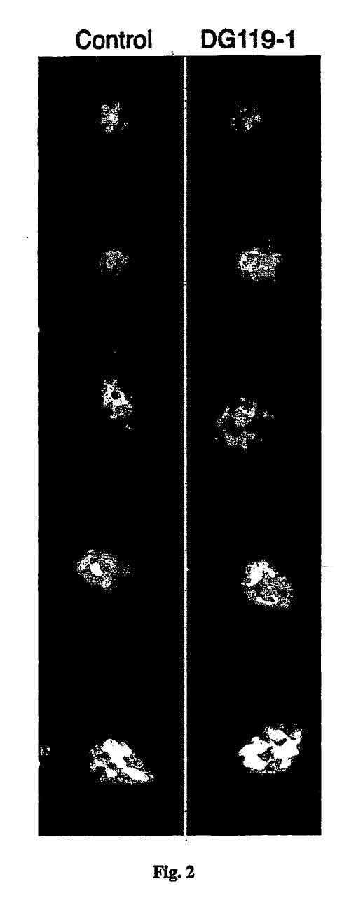

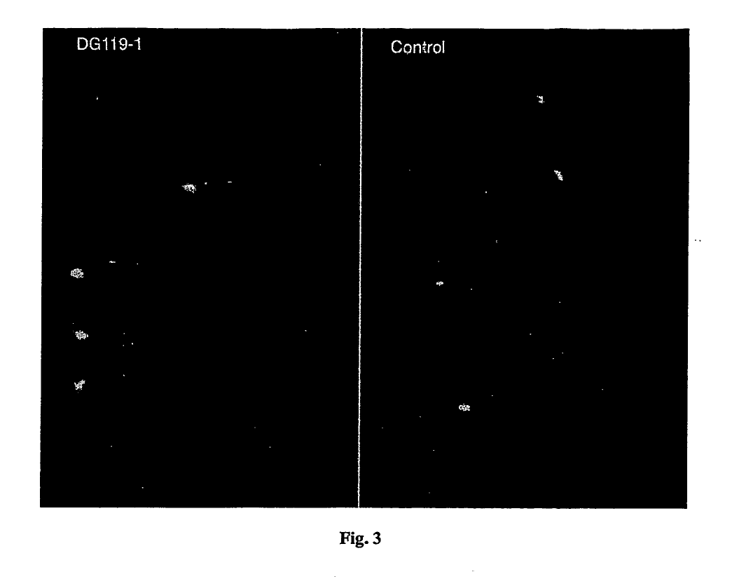

FIG. 1 is showing the internal organs of 17 d old fish embryos carrying a fluorescent protein. At this stage of development, the fish pancreas includes one bigger islet and several smaller islets, as marked by arrowheads in the figure. FIG. 2 clearly shows that the injection of DG119-1 antisense oligonucleotides increases the main islet size in these fish, and FIG. 3 shows the increased size of the small islets after DG119-1 antisense oligonucleotide injection.

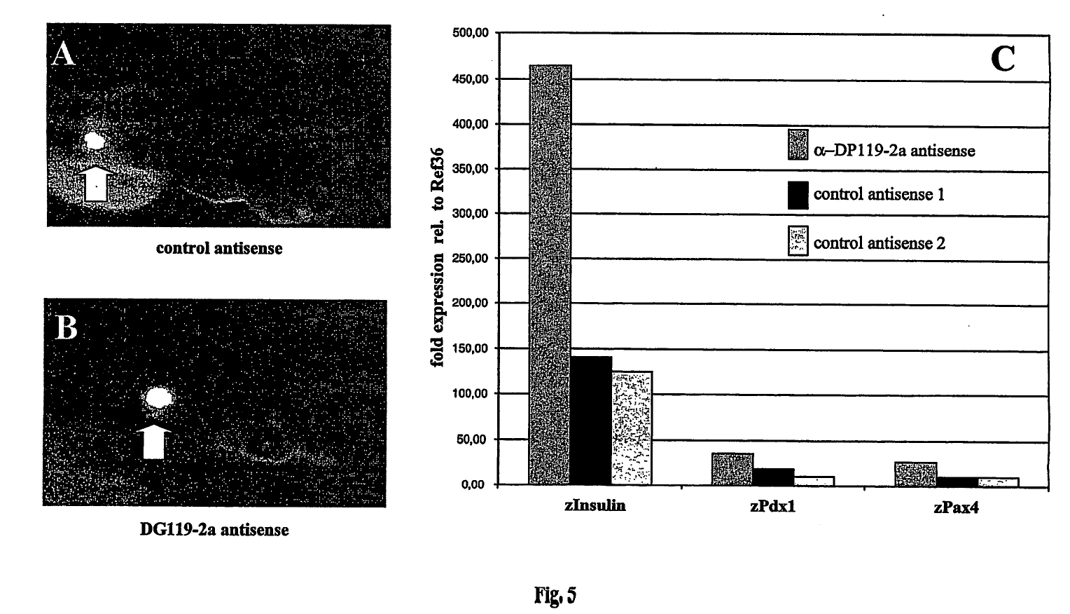

FIG. 5A shows the effect of loss-of-function of DG119-2A on the size of pancreatic islets. The islet size is significantly increased after the function of DG119-2A is lost due to injection of DG119-2 antisense oligonucleotides.

### Example 3

### Expression of DG119-1 and DG119-2 in Blood Vessels of the Pancreas

The full-length clone for mouse DG119-1 in the vector pCMV-SPORT6 was obtained from the Deutsches Ressourcenzentrum für Genomforschung GmbH (Berlin, Germany) and used as template for the synthesis of Digoxigenin (DIG)-labeled antisense RNA probes.

For obtaining the DG 119-2 probe for in situ hybridization, RNA was prepared from mouse embryonic stem cell by using Trizol, according to the manufactorer's instructions. cDNA was synthesized and PCR was performed for 35 cycles. The sequences of the PCR primers are as follows:

m1192 forward (SEQ ID NO: 5):
3′-gtgctgctgctgctggttttg-5′
m1192 reverse (SEQ ID NO: 6):
3′-ctgtgggctggggtattctgc-5′

The amplified PCR product was cloned into pCMV-Sport6 vector (purchased from Invitrogen) and used as template for synthesis of Digoxigenin (DIG)-labeled antisense RNA probes.

The in situ hybridization is described in Collombat et al., 2003, Genes Dev. 2003 17(20):2591-603. For DG119 RNA in situ hybridization on pancreas sections, mouse embryos of day 17 were collected and tissue isolated in ice-cold PBS. The tissue was fixed overnight in 4% paraformaldehyd containing 2 mM EGTA, incubated in 30% sucrose and embedded in tissue freezing medium (Leica). Tissue sections were applied to Super Frost Plus slides (Menzel-Gläser), dried at 42° C. for 30-60 min and stored at −80° C.

The hybridization of the DG119 antisense RNA to the mouse embryonic pancreas tissue can be localized using anti-digoxigenin antibodies. Defrosted sections were hybridized with Digoxigenin (DIG)-labeled antisenseRNA probes in a medium containing 50% formamid, 10% dextran sulphate, 1 mg/ml yeast tRNA, 0,02% BSA, 0.02% Ficoll, and 0.02% PVP at 65° C. overnight. The sections were washed successively in 50% formamide, 1×SSC at pH 5.3, 01% Tween-20 at 70° C. for 100 min, and 100 mM maleic acid, 150 mM NaCl, 0,1% Tween-20 (MABT) at pH 7.5 at room temperature for 1 hour. The sections were blocked in PBS containing 2% Blocking Reagent (Roche) and 20% goat serum for 1 hour. Anti-DIG antibody (1:2500) and guinea pig anti-insulin antibody (1:1000) were applied overnight in the same solution at room temperature. Tissues were washed thoroughly in MABT for 2 hours, and NTMT (100 mM NaCl, 100 mM Tris-HCl at pH 9.5, 50 mM MgCl₂, 1% Tween-20) for 20 min, stained in NBT/BCIP (Roche) in PBS and rinsed in NTMT.

For immunofluorescence detection of insulin and for staining the nucleus, the pancreas sections were rinsed in PBS for 10 min. Then, anti-guinea pig Cy3 conjugated secondary antibody (1:500) and DAPI stain (=4,6-diamidino-2-phenylindole; 1:10.000) were applied in PBS containing 2% goat serum for 30 min at room temperature. DAPI is a fluorochrome that binds to DNA and is used to stain the nucleus in fluorescence microscopy.The pancreas sections were rinsed in PBS for 20 min and covered with coverslips. Signals of the in situ hybridization were detected by immunofluorescence microscopy.

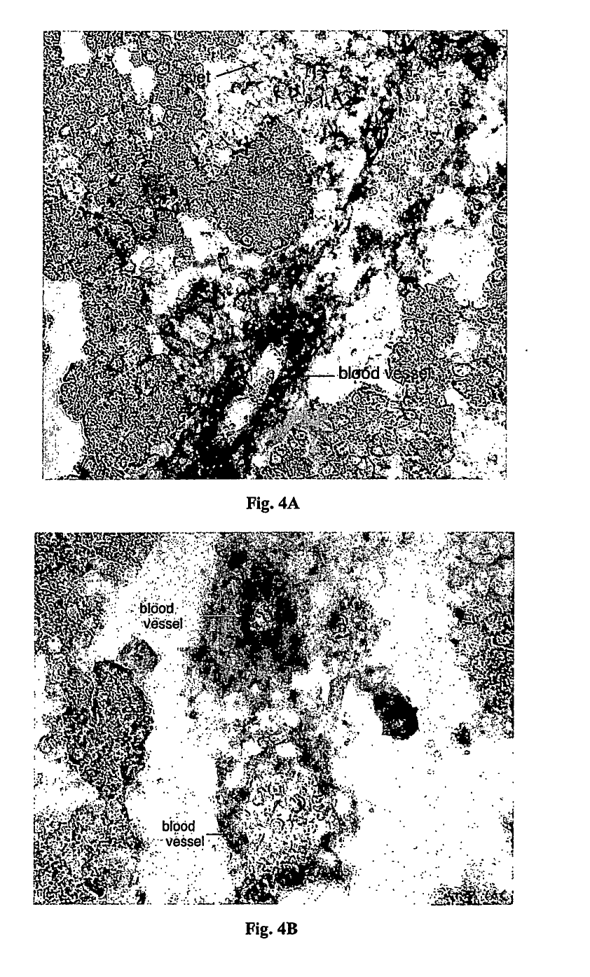

The results are presented in FIG. 4. FIG. 4 shows that both DG119-1 (FIG. 4A) are DG119-2 (FIG. 4B) are co-localized in pancreatic blood vessels.

### Example 4

### Loss-of-Function of DG119-2A Stimulates Insulin Expression in Zebrafish (Quantitative RT-PCR Analysis)

RNA was isolated from 20-30 fish embryos according to standard procedures, and quantitative RT-PCR (Taqman analysis) was performed according to standard procedures with primers specific for zebrafish (zf) Insulin1 (GenBank Accession Number NP_{—}571131), zebrafish (zf) Pdx1 (GenBank Accession Number NP_{—}571518), zebrafish (zf) Pax 4 (Accession Number Icl|ctg9534 Zebrafish shotgun assembly V2) and 18S RNA (Accession Number D751553).

FIG. 5B shows the effect of loss-of-function of DG119-2A on the expression levels of zebrafish insulin, pdx, and pax4. The expression of insulin is significantly enhanced (3-fold) upon inhibition of DG119-2A.

### Example 5

### Generation of DG119-1 Transgene Mammals (Mice)

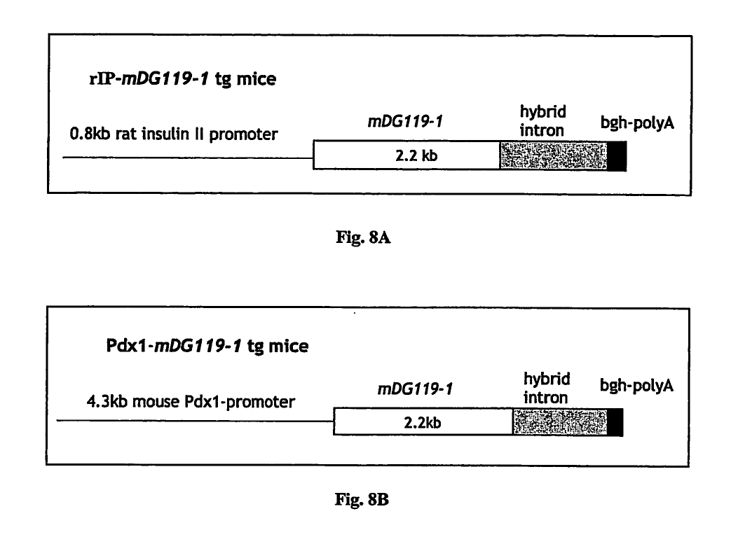

A complete mDG119-1 Open Reading Frame (ORF)-containing EST BC025654 (The I.M.A.G.E. Consortium (LLNL)) in pCMV-Sport6 vector (obtained from Invitrogen) was either cloned under the control of the rat insulin promoter II (Lomedico et al., (1979) Cell 18: 545-558) or under the control of the mouse PDX1 promoter (Gannon et al., (2001) Dev Biol. 238: 185-201) using the Gateway system (Invitrogen). For the structure of the transgenic constructs, see also FIG. 8A and FIG. 8B.

### Example 6

### Generation of rIP-mDG119-1 and Pdx1-mDG119-1 Transgenic Mice

Transgenic construct DNA (see Example 5) was injected into C57/BL6×CBA embryos (Harlan Winkelmann, Borchen, Germany) using standard techniques (see, for example, Brinster et al., (1985) Proc. Natl. Acad. Sci. USA 82: 4438-4442).

The mDG119-1 transgene (see Example 5) was expressed under the control of two different beta cell specific promoters: the rat insulin promoter II (Lomedico et al., supra) and the mouse Pdx-1 promoter (Gannon et al., supra) using techniques known to those skilled in the art (for example, see, Gunnig et al., (1987) Proc. Natl. Acad. Sci. USA 84: 4831-4835). Using this technique, several independent founder lines from each construct were generated.

Genotyping was performed by PCR using genomic DNA isolated from the tail tip (data not shown here).

### Example 7

### Islet Isolation from rIP-mDG119-1 and Pdx1-mDG119-1 Transgenic Mice

Islets were isolated using the standard collagenase procedure (Scharp et al., (1973) Transplantation 16: 686-689).

### Example 8

### mDG119-1 Expression Analysis via TaqMan Analysis

The expression of the mDG119-1 transgene in islets was monitored by TaqMan analysis. For this analysis, 25 ng cDNA derived from pancreatic islet RNA isolated from transgenic mice and their littermates and a mDG119-1 specifc primer/probe pair were used to detect endogenous as well as transgenic mDG119-1 expression:

mDG119-1 forward primer, SEQ ID NO: 7:
5′ GAG GAA AAT GAC ATA GAA GAG CAG C 3′,
mDG119-1 reverse primer, SEQ ID NO: 8:
5′ GCT GAT CTT CTA TCA GCA AGT CCA 3′,
mDG119-1 probe, SEQ ID NO: 9:
5′ CGA TGA GCT TTT CAG TGG CGA CAG TG 3′.

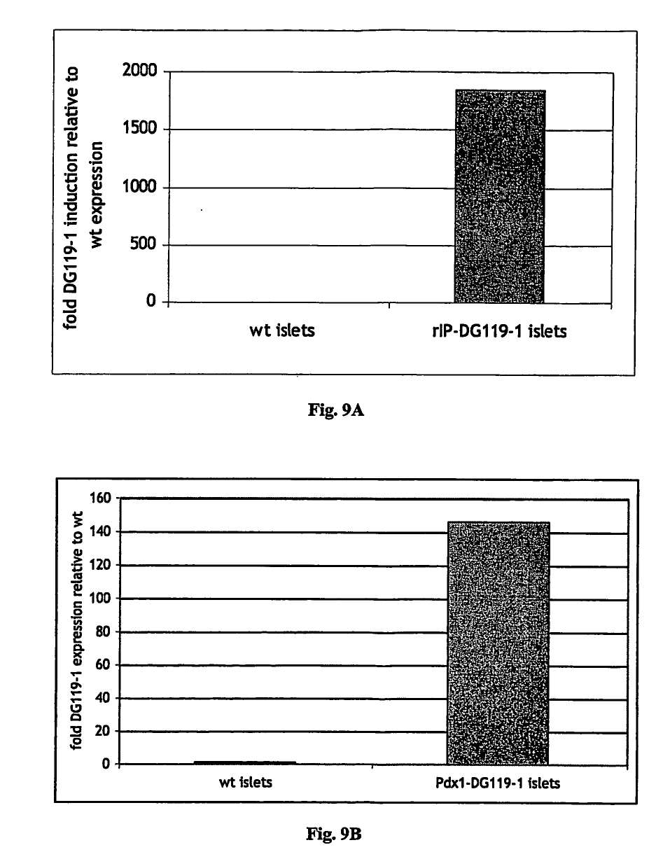

TaqMan analysis was performed using standard techniques known to those skilled in the art. Ectopic transgene expression was detected in 5 of 7 rIP-mDG119-1 transgenic founderlines and in 2 of 4 Pdx-1-mDG119-1 transgenic founderlines analysed. The two founderlines showing highest transgene expression levels were used for further analysis (see FIG. 9A (rIP-mDG119-1) and FIG. 9B (Pdx-1-mDG119-1)).

### Example 9

### Bodyweight, Body Length and NMR Analysis in mDG119-1 Transgenic Mice

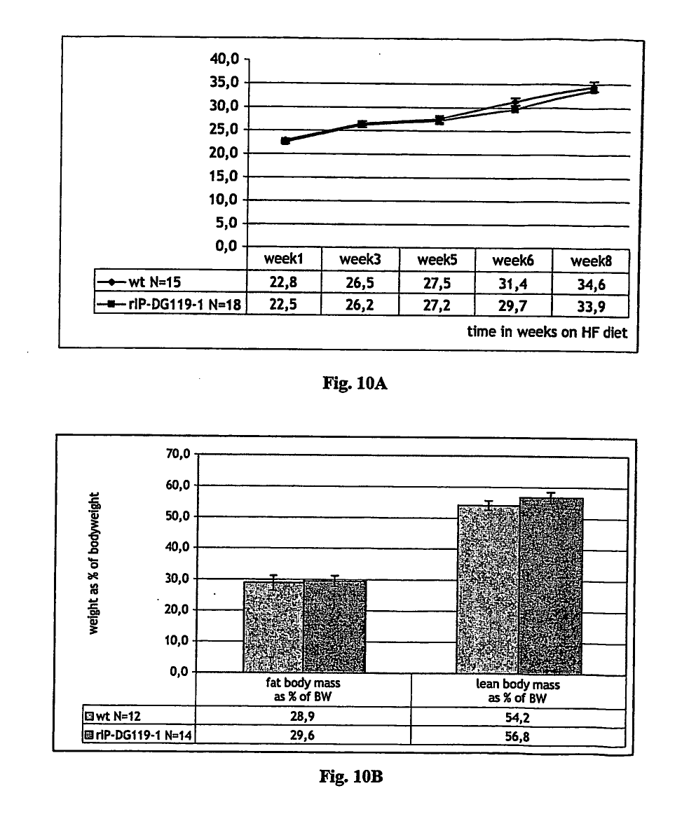

3 to 6 mice were housed per cage. Growth curves were generated by measuring the bodyweight of individual mDG119-1 transgenic mice and their wild-type littermates on a weekly basis using a standard balance. mDG119-1 transgenic mice show normal growth curves on high fat (HF) diet (see FIG. 10A (rIP-mDG119-1), data not shown for Pdx-1-mDG119-1).

On selected time points the lean and fat body mass was measured using non-invasive NMR analysis: individual mice were placed into a Bruker Minispec NMR machine (Bruker, USA) and the lean and body fat content was estimated. mDG119-1 transgenic mice showed normal lean and fat body mass after 8 weeks on high fat (HF) diet (see FIG. 10B (rIP-mDG119-1), data not shown for Pdx-1-mDG119-1).

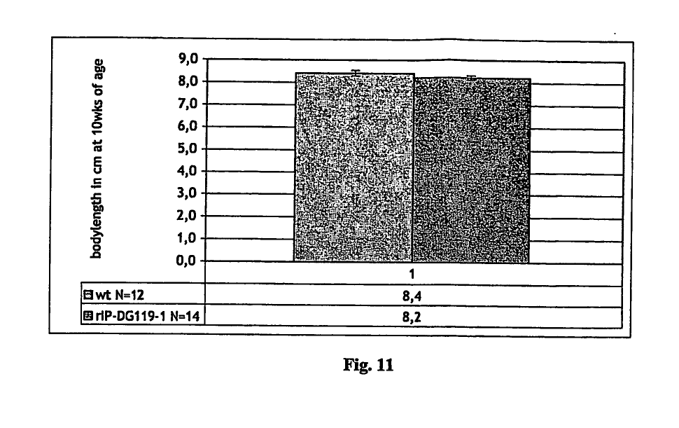

The body length was measured from nose to anus placing a ruler along the middle axis of the mouse. mDG119-1 transgenic mice show normal body length on HF diet compared to wild type mice (see FIG. 11 (rIP-mDG119-1), data not shown for Pdx-1-mDG119-1).

### Example 10

### Analytical Procedures Performed in mDG119-1 Transgenic Mice

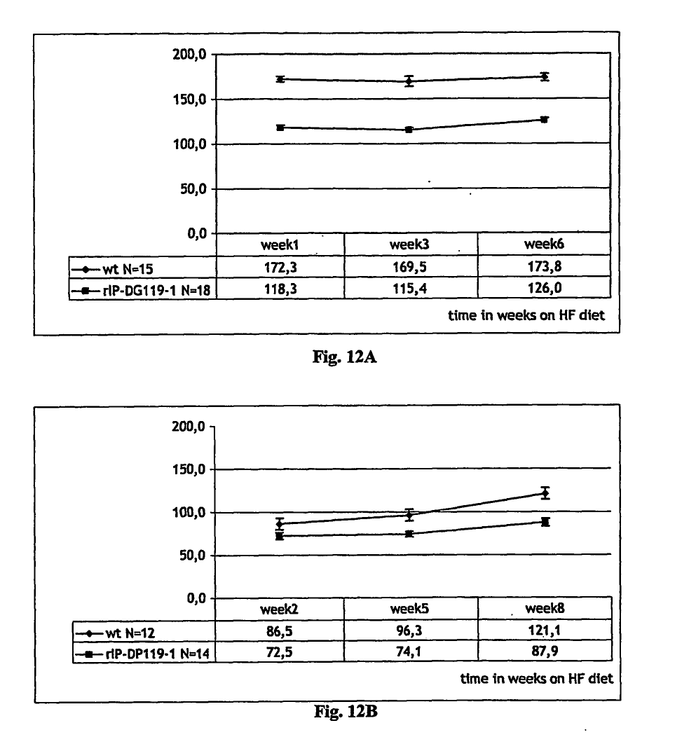

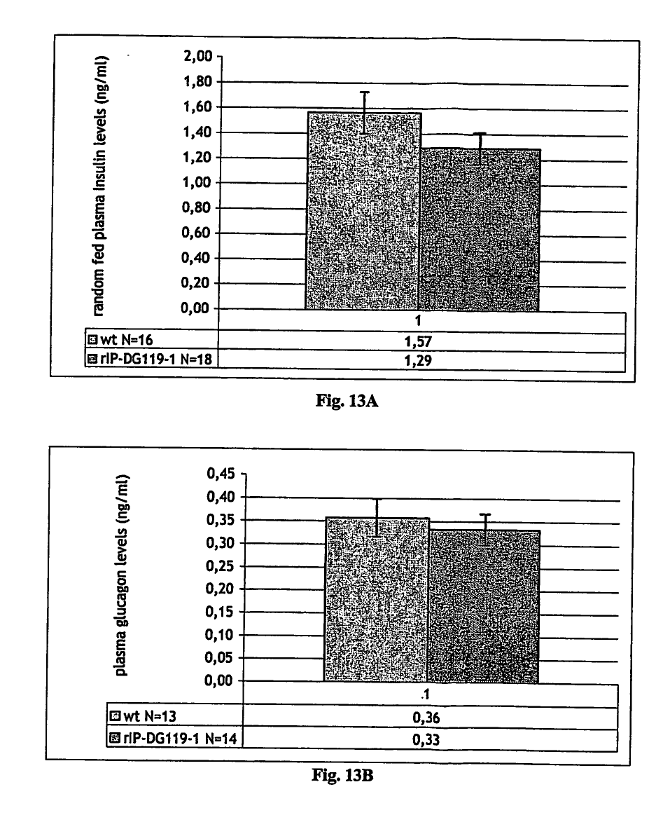

Metabolic blood parameters were determined using venous blood isolated from tail vein or via retroorbital bleeding. Blood glucose values were determined using One Touch blood glucose meters (LifeScan, Germany). For glucagon and insulin measurements, blood was collected in heparinized tubes (Sarstedt, Germany) and plasma was isolated via centrifugation. Plasma insulin and plasma glucagon levels were measured using commercially available ELISAs (Mercodia, Sweden & Bachem, Germany). mDG119-1 transgenic mice exhibit reduced random fed blood glucose levels (see FIG. 12A (rIP-mDG119-1), data not shown for Pdx-1-mDG119-1) and reduced starvation blood glucose levels (see FIG. 12B), compared to wild type mice. Further, normal steady state plasma insulin levels (FIG. 13A) and normal plasma glucagon levels (FIG. 13B) were measured in mDG119-1 transgenic mice, compared to wild type mice.

### Example 11

### Intraperitoneal Glucose Tolerance and Insulin Tolerance Tests in mDG119-1 Transgenic Mice

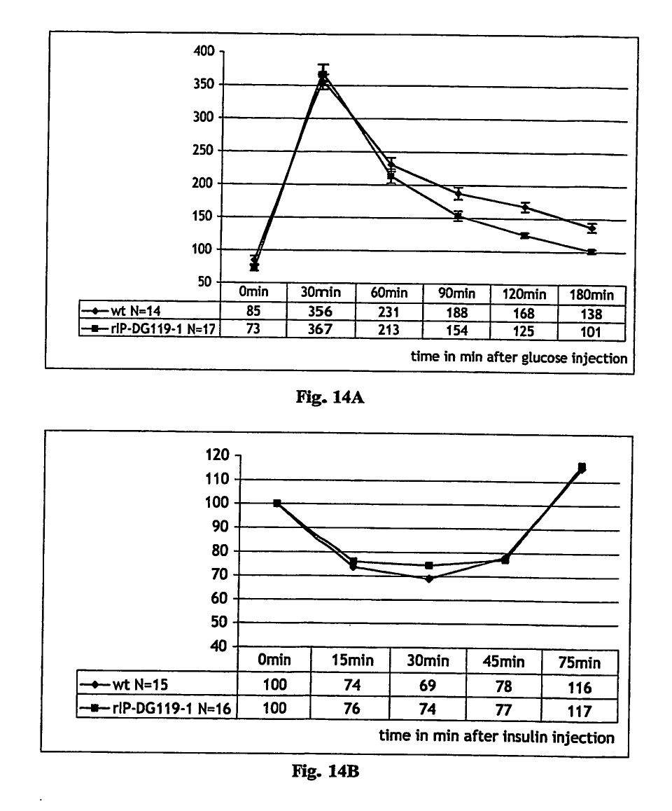

To perform glucose tolerance tests, mice were fasted over night for 16 h followed by intraperitoneal glucose injecton (1.5 g/kg bodyweight). Whole venous blood was obtained from the tail vein before and 30 min, 60 min, 90 min, 120 min and 180 min after injection. Blood glucose levels were determined as described (see Example 7). mDG119-1 transgenic mice demonstrate enhanced intraperitoneal glucose tolerance (see FIG. 14A (rIP-mDG119-1), data not shown for Pdx-1-mDG119-1).

To perform insulin tolerance tests, random fed mice were intraperitoneal injected with recombinant human Insulin (0.75 U/kg body weight) (Lilly, Germany). Whole venous blood was obtained from the tail vein before and 15 min, 30 min, 45 min and 75 min after injection. Blood glucose levels were determined as described (see Example 7). mDG119-1 transgenic mice show normal intraperitoneal insulin tolerance (see FIG. 14B).

### Example 12

### Measurement of Pancreatic Insulin Content in rIP-mDG119-1 Transgenic Mice

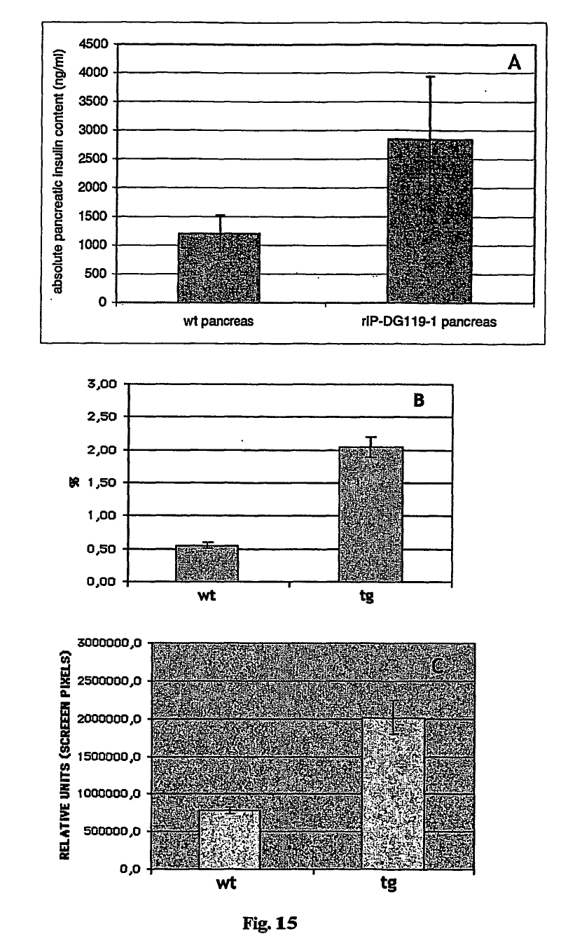

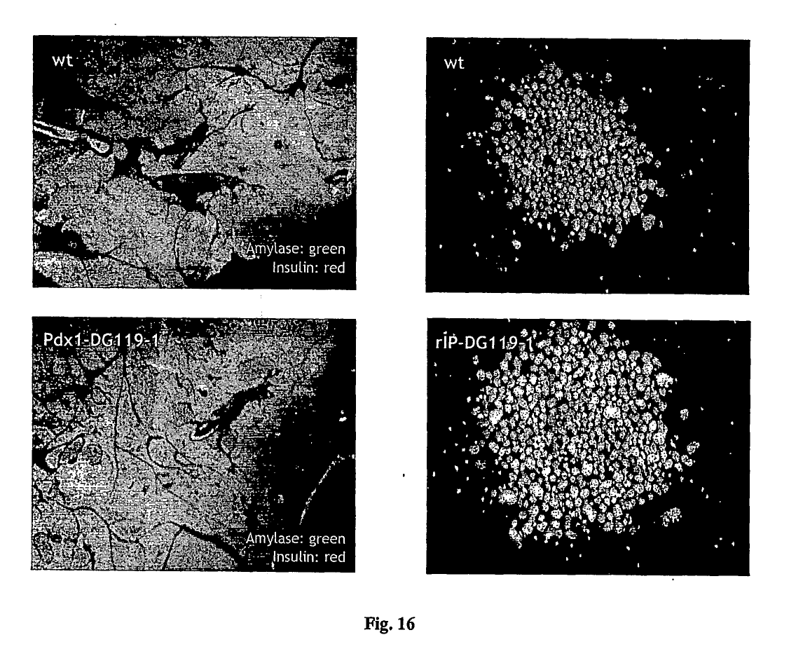

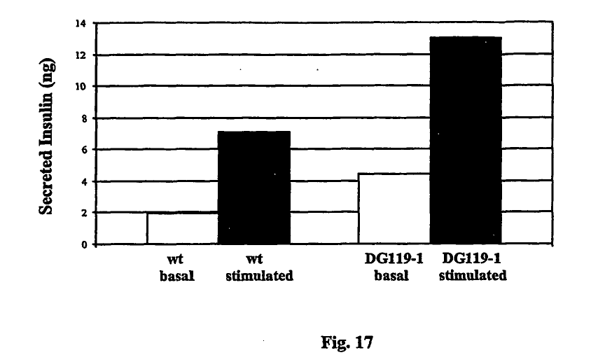

To measure pancreatic insulin content pancreata were dissected from mice and minced in acid alcohol using an electric homogenizer. After overnight incubation at 4° C. the homogenate was centrifuged and the supernatant was analysed for insulin content at a dilution of 1:1000 using a commercially available Insulin ELISA (Mercodia, Sweden). mDG119-1 transgenic mice show an increased pancreatic insulin content (see FIG. 15A).

### Example 13

### Morphometric Analysis of Pancreas Tissue in Pdx1-mDG119-1 Transgenic Mice

Pancreata of two wild type and two Pdx1-mDG119-1 transgenic 2 month old females were formalin-fixed, embedded in paraffin and cut to 5-μm sections according to standard histological techniques. From each pancreas, 6 sections spaced 100 μm from each other were taken for detection of insulin and amylase according to standard immunohistochemical protocol (i.e. Sund et al, (2001) Genes and Development 15: 1706-1715). The following antibodies were used at the indicated dilutions for immunohistochemistry: guinea pig anti-insulin (DAKO, 1:1000), rabbit anti-amylase (Sigma, 1:300), Cy-2 conjugated donkey anti-guinea pig IgG (Dianova), Cy-3 conjugated donkey anti-rabbit IgG (Dianova). Slides were mounted and random images of insulin and amylase stainings were taken using an Axiophot 2 compound microscope (Zeiss) equipped with epifluorescence and b/w Sensicam camera (INTAS). Image J program was used for double blind analysis of images. The area of insulin and amylase staining and the amount of islets in 96 images from wild type and Pdx1-mDG119-1 transgene was analysed. mDG119-1 transgenic mice show increased beta cell area (see FIG. 15B) and bigger islet size (see FIG. 15C) in comparison to wild type mice.

### Example 14

### Insulin Secretion in mDG119-1 Transgenic Islets In vitro

Mouse islets were isolated from three mDG119-1 transgenic animals and three respective wild type controls. 20 islets were distributed in 24-well plates, respctively. The islets were incubated for 1 hour at 37° C. in 1 ml 2.8 mM glucose in KRBH (Krebs Ringer Buffer HEPES). Subsequently the supernatant was discarded, and the islets were incubated again for 1 hour at 37° C. in 1 ml 2.8 mM glucose in KRBH (Krebs Ringer Buffer HEPES). Following incubation, the medium was changed and aliquots of the supernatant were taken for further analysis. The islets were subsequently incubated for 15 min (minutes 1-15) at 37° C. in 1 ml 16.7 mM glucose in 1 ml KRBH (Krebs Ringer Buffer HEPES). The medium was replaced and aliquots of the supernatant were taken for further analysis. The procedure was repeated three times under the same conditions (minutes 16-30, 3145, 46-60), followed by incubation for 15 min at room temperature in 1 ml 16.7 mM glucose, 30 mM KCl in KRBH (Krebs Ringer Buffer HEPES). Aliquots of the supernatant were taken for further analysis. Afterwards the islets were incubated over night at 4° C. in 1 ml acid/ethanol (70% EtOH, 0.2 N HCl) and aliquots of the supernatant were taken. The aliquots were centrifuged at 3000 rpm in a desktop centrifuge for 5 minutes prior to aliquoting to a new tube and freezing at −20° C. for storage until measurement. The acid/ethanol samples were centrifuged at 13200 rpm for 10 min before re-aliquoting and freezing. For measurement, 300 μl aliqouts were used per sample. The aliquots were stored at −20° C.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. Use of a DG119-1 product and/or a DG119-1 agonist and/or of a DG119-2 antagonist for the manufacture of a medicament to stimulate and/or induce the differentiation or development of insulin producing cells from progenitor cells.

2. The use of claim 1, wherein the progenitor cells are stem cells.

3. The use of claim 1, wherein the stem cells are embryonic or somatic stem cells.

4. The use of claim 1, wherein the stem cells are of mammalian origin, preferably of human origin with the proviso that the use of human embryos is excluded.

5. The use of claim 1, wherein the progenitor cells have been transfected with a pancreatic gene, particularly the Pax4 gene.

6. Use of a DG119-1 product and/or a DG119-1 agonist and/or of a DG119-2 antagonist for the manufacture of a medicament to promote the protection survival and/or regeneration of insulin producing cells.

7. The use of claim 6, wherein the insulin producing cells are beta-cells.

8. The use of claim 6, wherein the insulin producing cells are of mammalian origin, preferably of human origin, with the proviso that the use of human embryos is excluded.

9. The use of claim 6, wherein the insulin producing cells have been transfected or transduced with a pancreatic gene, particularly the Pax4 gene.

10. The use of claim 1 for the prevention or treatment of a disease caused by, accompanied by or associated with impaired beta-cell function.

11. The use of claim 10 for the treatment of beta-cell degeneration in patients suffering from diabetes type I, LADA, or progressed diabetes type II.

12. The use of claim 10 for the prevention of beta-cell degeneration in patients at risk to develop beta-cell degeneration, like for example but not limited to patients suffering from diabetes type I or II, or LADA in early stages.

13. The use of claim 1, wherein the active ingredient is administered to a patient
(i) as a pharmaceutical composition e.g. enterally, parenterally or topically directly to the pancreas,
(ii) via implantation of active ingredient expressing cells, and/or
(iii) via gene therapy.

14. The use of claim 13, wherein the active ingredient is administered in combination with another pharmaceutical composition useful to treat beta-cell degeneration, for example but not limited to hormones, growth factors, or immune modulating agents.

15. The use of claim 1, wherein the DG119-1 product is a protein including purified natural, synthetic or recombinant DG119-1 and variants thereof.

16. The use of claim 15 wherein variants are selected from insertion, substitution, deletion variants and/or chemically modified derivatives, for example but not limited to hybrids of DG119-1 and other proteins.

17. The use of claim 15, wherein the DG119-1 product is selected from proteins or peptides substantially homologous to the human DG119-1 protein having the amino acid sequence published as GenBank Accession Number XP_{—}034000.

18. The use of claim 1, wherein the DG119-1 product is a nucleic acid, e.g. RNA and/or DNA encoding a DG119-1 protein product.

19. The use of claim 1, wherein the DG119-2 antagonist is selected from DG119-2 fragments, modified DG119-2 proteins, antibodies, and biologically active nucleic acids.

20. The use of claim 1, wherein an effective amount of cells treated in vitro with the active ingredient are transplanted to a patient in need.

21. The use of claim 1, comprising modifying DG119-1 and/or DG119-2 expression, wherein cells from a patient in need that have been modified to produce and secreted DG119-1 protein product and/or a DG119-1 agonist and/or a DG119-2 antagonist in vitro are re-implanted into the patient and/or wherein cells of a patient in need are modified to produce and secrete a DG119-1 protein product and/or a DG119-1 agonist and/or a DG119-2 antagonist in vivo.

22. The use of claim 1 in combination with at least one other pharmaceutical agent.

23. The use of claim 22 in combination with at least one other pharmaceutical agent suitable for the treatment or prevention of pancreatic diseases and/or obesity and/or metabolic syndrome.

24. The use of claim 23 in combination with at least one other pharmaceutical agent suitable for stimulating and/or inducing the differentiation or development of insulin producing cells from progenitor cells.

25. The use of claim 22 in combination with at least one other pharmaceutical agent which has an immunosuppressive activity.

26. A method for differentiating or regenerating cells into functional pancreatic cells, the method comprising: (a) cultivating cells capable of being differentiated or regenerated into pancreatic cells in the presence of an effective amount of a DG119-1 product and/or a DG119-1 agonist and/or a DG119-2 antagonist in vitro (b) allowing the cells to develop, to differentiate and/or to regenerate at least one pancreatic function; and (c) optionally preparing an effective amount of the differentiated or regenerated pancreatic cells for transplantation into a patient in need thereof.

27. The method of claim 26, wherein the patient in need is a human individual.

28. The method of claim 26, wherein the patient in need has (a) functionally impaired, (b) reduced numbers and/or (c) functionally impaired and reduced numbers of pancreatic cells.

29. The method of claim 26, wherein said patient in need is a type I diabetic patient or type II diabetic patient or LADA patient.

30. The method of claim 26, wherein the pancreatic cells are insulin-producing cells.

31. The method of claim 26, wherein the pancreatic cells are beta-cells of the pancreatic islets.

32. The method of claim 26, wherein the cells in step (a) are selected from embryonic stem cells, adult stem cells, somatic stem cells or progenitor cells, preferably derived from pancreatic tissue.

33. The method of claim 26, wherein the cells in step (a) are of mammalian origin, preferably human origin, with the proviso that the use of human embryos is excluded.

34. The method of claim 26, wherein the cells in step (b) have at least one pancreatic function selected from insulin production in response to glucose and expression of glucagon.

35. A method for differentiating or regenerating cells into functional pancreatic cells, the method comprising: preparing an effective amount of a DG119-1 product or a DG119-1 agonist and/or a DG119-2 antagonist or of cells capable of expressing a DG119-1 product and/or a DG119-1 agonist and/or a DG119-2 antagonist for administration to a patient in need thereof.

36. The method of claim 35, wherein the active ingredient is a protein product.

37. The method of claim 36, wherein the active ingredient is a nucleic acid.

38. The method of claim 37, wherein cells have been modified to produce and secrete a DG119-1 product and/or a DG119-1 agonist and/or a DG119-2 antagonist and are prepared for transplantation into a suitable location in the patient.

39. A cell preparation comprising functional pancreatic cells treated with an active ingredient selected from a DG119-1 product and/or a DG119-1 agonist and/or a DG119-2 antagonist obtainable by the method of claim 26.

40. A cell preparation comprising cells expressing an active ingredient selected from a DG119-1 product and/or a DG119-1 agonist and/or a DG119-2 antagonist obtainable by the method of claim 35.

41. The preparation of claim 39, which is a pharmaceutical composition.

42. The preparation of claim 39 for the treatment or prevention of pancreatic diseases, particularly diabetes.

43. The preparation of claim 39 for administration by transplantation or for use in a medical device.

44. The preparation of claim 39, which contains pharmaceutically acceptable carriers, diluents, and/or additives.

45. The preparation of claim 39, which is a diagnostic composition.

46. The preparation of claim 39, which is a therapeutic composition.

47. The preparation of claim 39 for the manufacture of an agent for the regeneration of pancreatic tissues or cells, particularly pancreatic beta cells.

48. The preparation of claim 39 for application in vivo.

49. The preparation of claim 39 for application in vitro.

50. A method for identifying and/or characterizing compounds capable of modulating the differentiation or regeneration of cells into functional pancreatic, particularly insulin-producing cells comprising:
contacting a compound to be tested with cells under conditions wherein the cells are capable of being differentiated or regenerated into functional pancreatic cells in the presence of DG119-1, a DG119-1 agonist and/or a DG119-2 antagonist and determining the effect of the compound on the differentiation process.

51. The method of claim 50 comprising transfecting the cells with a DNA construct containing a reporter gene under regulatory control of a gene involved in beta-cell differentiation, contacting said transfected cells with a compound to be tested and determining the activity of the reporter gene.

52. A method for identifying and/or characterizing compounds capable of modulating the differentiation or regeneration of cells into functional pancreatic, particularly insulin-producing cells comprising:
contacting a compound to be tested with cells under conditions wherein the cells are capable of being differentiated or regenerated into functional pancreatic cells and determining the effect of the compound on the expression of DG119-1 and/or DG119-2.

53. Use of a preparation of cells expressing an active ingredient selected from a DG119-1 product and/or a DG119-1 agonist and/or a DG119-2 antagonist for the treatment and prevention of diabetes.

54. The use of claim 53 for inducing the regeneration of pancreatic cells.

55. The use of claim 54, wherein pancreatic cells are beta-cells of the islets.

56. Use of a preparation of cells treated with an active ingredient selected from a DG119-1 product and/or a DG119-1 agonist and/or a DG119-2 antagonist for the treatment and/or prevention of diabetes.

57. The use of claim 56 wherein the cells are differentiated progenitor cells capable of insulin production.


SEQUENCE LISTING (13 sequences)

SEQ ID NO: 1 - protein, 719 aa, Danio rerio
MTEMKIWCVLLMAFALTSAAPKSHLRLEEKTKDNNDTLQVEIDNQEHILSQLLGDYDKVKALSEGSDCGCKCVVRPLSASACQRIREGHATPQDFYTVETITSGPHCKCACIAPPSALNPCEGDFRLKKLRQAGKDNIKLSTILELLEGSFYGMDLLKLHSVTTKILDRMDTIEKMVLNNQTEEKLNTISTSPNPQLSTSSPTTLPSVIQEKSTSLRQQNDEAAAFQHMESKYEEKFVGDILNSGSDLNKATTALQEQEQQGRKKQPKITVRGITYYRSDPVDEMDSEKNLKETSASSVTQTGALIKEHLKASTQSTLNTLTPSPTSHSNALTVTESSVGINAHKGEVTTIVMTASVTGSKTDSVTDLTQLSPRVRETLTTTRTTTKTATTSQPVKRKYSISWDEEEEAVVPEQVEEEKAVKPVVEDKVGEEPQRKPGTAHHQAKTISTVKQQIKFSLGMCKDTLATISEPITHNTYGRNEGAWMKDPLDQDDKIYVTNYYYGNNLLEFRNIDVFKQGRFTNSYKLPYNWIGTGHVVYKGAFYYNRAFSRDIIKFDLRLRYVAAWTMLHDAVFENDDVSSWRWRGNSDMDLAIDESGLWVIYPALDDEGFLQEVIVLSRLNPTDLSMKRETTWRTGLRRNRYGNCFIVCGVLYATDSYNQQDTNLSYAFDTHTNTQVIPHLPFSNNYTYVTQIDYNPKERVLYAWDNGHQVTYNVQFAY

SEQ ID NO: 2 - protein, 594 aa, Danio rerio
  misc_feature (198)..(198): Xaa can be any naturally occurring amino acid
MGLLLYIFCCVFCLTRANVEQQATDNTDNRATLEDEMDNQENILTQLIGDYDKVKTLSEGSDCQCKCVVRPMSRSACKRIEEAQAKIEDFYTVEPVTAGPNCKKCACIAPPSALNPCEGDFRFKKLQKTGQYDIKLSNIMDLLEERVDNIEKGEKGQGKGARSNQRQEKKKRLSVVCWSLHCRRTQQRLLLTLRYRCXSVLEPSLQKNAAAAFAHTEVQMQQFIPDQRKYEEKFVGNQGPSKPVLKKSKSEGQEEQHKPAKTKADAKNMSLRSMTFYKANRMEDSEGEERDLIIEDQLHKQGLNTPVTTPEATVTVTQSTTINLNTQNFTTARMSNVTKQTQGQSVKAMMSSTITTERPTMPTSTTSTSTMTPGTNTTTIATPLVVPKQLASVTVGQVSNSYKLPYNWIGTGHVVYSGSFFYNRAFSRDIIRFDLRLRYVAAWTTLHDAILEEEEAPWTWGGHSDIDFSVDESGLWLVYPALDDEGFHQEVIILSKLRASDLQKEKSWRTGLRRNYYGNCFVICGVLYAVDSFERTHANISYAFDTHTHTQMIPRLPFINNYTYTTQIDYNPKERMLYAWDNGHQVTYDVIFAY

SEQ ID NO: 3 - protein, 146 aa, Danio rerio
MWRIVELVACLLMMSSHVSSQSKIFGEEQVRMTSEGSDCRCKCIMRPLTRDACARLRTGSVRVEDFYTVETVSSGADCKCSCTAPPSSLNPCENEWKREKLKKQAPELLKLQSMVDLLEGTLFSMDLLKVHSYINKVVSQMNNLEE

SEQ ID NO: 4 - protein, 287 aa, Danio rerio
  misc_feature (103)..(103): Xaa can be any naturally occurring amino acid
MWIYASVLTYLLLLTRDARSLSKIFGEPEPVKMISEGSDCRCKCVMRPLSIEACSRLRDGSLRVDDFYTVETVSSGSDCKCSCTAPPSSLNPCENEWRTEKLXKQAPELLKLHSMVDLLEGTLYSMDLMKVHAYMNKVVSQMNTLEEVMTIKTNLTRENEFVRDSVVNLSNQLKRYENYSDIMVSIKKEISSLGLQLLQKDAASDSKAQVGTESKKSKEAIKPPNKKPPAVKPPPKQPKEKPVKPKKEAPAKAAKPAKPDPTTKTKTSVHQTGVIRGITYYKASKSE

SEQ ID NO: 5 - DNA, 21 bp, Mus musculus
gttttggtcgtcgtcgtcgtg

SEQ ID NO: 6 - DNA, 21 bp, Mus musculus
cgtcttatggggtcgggtgtc

SEQ ID NO: 7 - DNA, 25 bp, Mus musculus
gaggaaaatgacatagaagagcagc

SEQ ID NO: 8 - DNA, 24 bp, Mus musculus
gctgatcttctatcagcaagtcca

SEQ ID NO: 9 - DNA, 26 bp, Mus musculus
cgatgagcttttcagtggcgacagtg

SEQ ID NO: 10 - protein, 746 aa, Mus musculus
MAYPLPLVLCFALVVAQVWGSTTPPTGTSEPPDVQTVEPTEDDILQNEADNQENVLSQLLGDYDKVKAVSEGSDCQCKCVVRPLGRDACQRINQGASRKEDFYTVETITSGSSCKCACVAPPSAVNPCEGDFRLQKLREADSRDLKLSTIIDMLEGAFYGLDLLKLHSVTTKLVGRVDKLEEEVSKNLTKENEQIKEDVEEIRTELNKRGKENCSDNTLESMPDIRSALQRDAAAAYAHPEYEERFLQEETVSQQINSIELLRTQPLVPPAAMKPQRPLQRQVHLRGRLASKPTVIRGITYYKAKVSEEENDIEEQHDELFSGDSGVDLLIEDQLLRQEDLLTSATRRPATTRHTAAVTTDASIQAAASSSEPAQASASASSFVEPAPQASDRELLATPQTTTVFPEPTGVMPSTQVSPTTVAHTAVQPLPAMVPGDIFVEALPLVPLLPDTVGTDMPEEEGTAGQEATSAGPILSPEEEDDIRNVIGRCKDTLSTITGPTTQNTYGRNEGAWMKDPLAKDDRIYVTNYYYGNTLVEFRNLENFKQGRWSNSYKLPYSWIGTGHVVYNGAFYYNRAFTRNIIKYDLKQRYVAAWAMLHDVAYEEATPWRWQGHSDVDFAVDENGLWLIYPALDDEGFNQEVIVLSKLNAVDLSTQKETTWRTGLRRNFYGNCFVICGVLYAVDSYNQRNANISYAFDTHTNTQIVPRLLFENEYSYTTQIDYNPKDRLLYAWDNGHQVTYHVIFAY

SEQ ID NO: 11 - protein, 861 aa, Homo sapiens
MAKPRLLVLYFALIVVPAWVSSIVLTGTSEPPDAQTVAPAEDETLQNEADNQENVLSQLLGDYDKVKAMSEGSDCQCKCVVRPLGRDACQRINAGASRKEDFYTVETITSGSSCKCACVAPPSALNPCEGDFRLQKLREADSQDLKVGPGMGQCLGREGTFEIHKSGKAMVEDSKPFEEGLSHFLTQTFRKAECTYTIVLAYIPVYTNVFLTATSQFLASGFPVEPPLSTIIDMLEGAFYGLDLLKLHSVTTKLVGRVDKLEEMLEGAFYGLDLLKLHSVTTKLVGRVDKLEEEVSKNLTKENEQIKEDMEEIRTEMNKRGKENCSENILDSMPDIRSALQRDAAAAYAHPEYEERFLQEETVSQQINSIELLQTRPLALPEVVKSQRPLQRQVHLRGRPASQPTVIRGITYYKAKVSEEENDIEEQQDEFFSGDNGVDLLIEDQLLRHNGLMTSVTRRPAATRQGHSTAVTSDLNARTAPWSSALPQPSTSDPSIANHASVGPTLQTTSVSPDPTRESVLQPSPQVPATTVAHTATQQPAAPAPPAVSPREALMEAMHTVPVPPTTVRTDSLGKDAPAGWGTTPASPTLSPEEEDDIRNVIGRCKDTLSTITGPTTQNTYGRNEGAWMKDPLAKDERIYVTNYYYGNTLVEFRNLENFKQGRWSNSYKLPYSWIGTGHVVYNGAFYYNRAFTRNIIKYDLKQRYVAAWAMLHDVAYEEATPWRWQGHSDVDFAVDENGLWLIYPALDDEGFSQEVIVLSKLNAADLSTQKETTWRTGLRRNFYGNCFVICGVLYAVDSYNQRNANISYAFDTHTNTQIVPRLLFENEYSYTTQIDYNPKDRLLYAWDNGHQVTYHVIFAY

SEQ ID NO: 12 - protein, 681 aa, Mus musculus
MEAAAVLPRYLQLRLLLVLLLLVLLRAGPVWPDSKVFSDLDQVRMTSEGSDCRCKCIMRPLSKDACSRVRSGRARVEDFYTVETVSSGADCRCSCTAPPSSLNPCENEWKMEKLKKQAPELLKLQSMVDLLEGALYSMDLMKVHAYIQKVASQMNTLEESIKANLSLENKVVKDSVHHLSEQLKSYENQSAIMMSIKKELSSLGLQLLQRDAAAVPATAPASSPDSKAQDTAGGQGRDLNKYGSIQKSFSDKGLAKPPKEKLLKVEKLRKESIKGRIPQPTARPRALAQQQAVIRGFTYYKAGRQEARQEARQEAPKAAADSTLKGTSWLEKLPPKIEAKLPEPNSAKHDDVRLQASEGGNLTPDITTTTTSTSSSTTTTTGTTSTTSTTSTTSTTTPSPITTPWPTEPPLHPEVPSQGREDSCEGTLRAVDPPVKHHSYGRHEGAWMKDPAALDDRIYVTNYYYGNSLVEFRNLENFKQGRWSNMYKLPYNWIGTGHVVYQGAFYYNRAFTKNIIKYDLRQRFVASWALLPDVVYEDTTPWKWRGHSDIDFAVDESGLWVIYPAVDEHDETQHEVIVLSRLDPADLSVHRETTWKTRLRRNSYGNCFLVCGILYTVDTYNQHEGQVAYAFDTHTGTDAHPQLPFLNEYSYTTQVDYNPKERVLYAWDNGHQLTYTLHFVV

SEQ ID NO: 13 - protein, 704 aa, Homo sapiens
MAAAALPPRPLLLLPLVLLLSGRPTRADSKVFGDLDQVRMTSEGSDCRCKCIMRPLSKDACSRVRSGRARVEDFYTVETVSSGTDCRCSCTAPPSSLNPCENEWKMEKLKKQAPELLKSIKANLSRENEVVKDSVRHLSEQLRHYENHSAIMLGIKKELSRLGLQLLQKDAAAAPATPATGTGSKAQDTARGKGKDISKYGSVQKSFADRGLPKPPKEKLLQVEKLRKESGKGSFLQPTAKPRALAQQQAVIRGFTYYKAGKQEVTEAVADNALQGTSWLEQLPPKVEGRSNSAEPNSAEQDEAEPRSSERVDLASGTTHLILPPHSLHHHSTPVLATPAPFHLQCHNKPVPSPRRWQTTPSRALPGWSNCRPRWRAGPTPQSPTPQSRMRLSPGPPSEWTWLLAPHFNPCHHHHRHPHPQPPTTSLLPTEPPSGPEVSSQGREASCEGTLRAVDPPVRHHSYGRHEGAWMKDPAARDDRIYVTNYYYGNSLVEFRNLENFKQGRWSNMYKLPYNWIGTGHVVYQGAFYYNRAFTKNIIKYDLRQRFVASWALLPDVVYEDTTPWKWRGHSDIDFAVDESGLWVIYPAVDDRDEAQPEVIVLSRLDPGDLSVHRETTWKTRLRRNSYGNCFLVCGILYAVDTYNQQEGQVAYAFDTHTGTDARPQLPFLNEHAYTTQIDYNPKERVLYAWDNGHQLTYTLHFVV